(19) United States
(12) Patent Application Publication
(10) Pub. No.: US 2013/0230453 A1
(43) Pub. Date: Sep. 5, 2013

(54) DIAGNOSIS AND TREATMENT OF BRAIN TUMORS

(71) Applicants: Peter John Wookey, North Melbourne (AU); Sebastian Furness, Coburg North (AU); Terrance Johns, Box Hill North (AU)

(72) Inventors: Peter John Wookey, North Melbourne (AU); Sebastian Furness, Coburg North (AU); Terrance Johns, Box Hill North (AU)

(73) Assignee: Welcome Receptor Antibodies Pty Ltd, North Melbourne, Victoria (AU)

(21) Appl. No.: 13/806,985

(22) PCT Filed: Jul. 1, 2011
(86) PCT No.: PCT/AU11/00835

§ 371 Date: May 14, 2013

(30) Foreign Application Priority Data
Jul. 2, 2010 (AU) 2010902958

(51) Int. Cl. A61K 49/00; A61K 45/06; A61K 45/00; G01N 33/74; A61K 31/713

(52) U.S. Cl. CPC A61K 49/0002; G01N 33/74; A61K 31/713; A61K 45/00; A61K 45/06
USPC 424/1.11; 435/7.23; 424/9.1; 424/85.4; 424/133.1; 514/44 A

(57) ABSTRACT

The present invention relates to methods for the localisation, diagnosis, prognosis and/or prediction of therapeutic outcome of cancer, as well as methods for treating or preventing cancer. In particular, the present invention relates to methods for the localisation, diagnosis, prognosis and/or prediction of therapeutic outcome of brain tumors expressing calcitonin receptor, as well as the treatment and prevention of brain tumors by targeting calcitonin receptor expressing brain tumour cells.

### FIELD OF THE INVENTION

The present invention relates to methods for the localisation, diagnosis, prognosis and/or prediction of therapeutic outcome of cancer, as well as methods for treating or preventing cancer. In particular, the present invention relates to methods for the localisation, diagnosis, prognosis and/or prediction of therapeutic outcome of brain tumors, as well as the treatment and prevention of brain tumors.

### BACKGROUND OF THE INVENTION

In 2008, there were over 21,000 new cases of brain and other nervous system tumors in the United States, and over 13,000 deaths. Common types of brain tumor are astrocytoma (including low-grade glioma, high-grade astrocytoma, and glioblastoma and glioblastoma multiforme), meningioma, oligodendroglioma, medulloblastoma, ependymoma and brain stem glioma.

Glial tumors, the most prevalent and morbid of which are high-grade astrocytoma and the aggressive glioblastoma multiforme, are the most common brain tumors in adults. They are also among the least treatable cancers, with a 3 year survival after initial diagnosis of <10% for tumors initially diagnosed at the grade 4 (glioblastoma) stages. The current treatments of glioma and glioblastoma achieve only palliation and short-term increments in survival. They include surgical resection, following which ultimate recurrence rates are over 90%, as well as radiation therapy, and chemotherapies.

Nitrosourea chemotherapeutic agents have normally been used in the treatment of brain tumors. The key property of these compounds is their ability to cross the blood-brain barrier. 1-3-bis-2-chloroethyl-1-nitrosourea (BCNU, also known as Carmustine) was the first of these to be used clinically. While the use of BCNU in combination with surgery and/or radiation treatment has been shown to be beneficial, it has not cured glioblastoma multiforme brain tumors. Additionally, complications with prolonged nitrosourea treatment include pulmonary fibrosis, hepatic toxicity, renal failure and cases of secondary tumors associated with nitrosourea treatment.

While a treatment regimen of surgery, radiation therapy and chemotherapy offers the opportunity for a modestly increased lifespan for patients with a grade IV astrocytoma brain tumor, the risks associated with each method of treatment are many. The benefits of treatment are minimal, and treatment can significantly decrease the quality of the patient's remaining lifespan. Accordingly, there remains a need in the art for methods of treating brain cancers that overcome the disadvantages of the existing approaches.

Imaging plays a central role in the diagnosis of brain tumors. Early imaging methods, invasive and sometimes dangerous, such as pneumoencephalography and cerebral angiography, have been abandoned in recent times in favour of non-invasive, high-resolution modalities, such as computed tomography (CT) and especially magnetic resonance imaging (MRI).

The definitive diagnosis of brain tumor can only be confirmed by histological examination of tumor tissue samples obtained either by means of brain biopsy or open surgery. The histological examination is essential for determining the appropriate treatment and the correct prognosis. This examination, performed by a pathologist, typically has three stages: interoperative examination of fresh tissue, preliminary microscopic examination of prepared tissues, and follow-up examination of prepared tissues after immunohistochemical staining or genetic analysis.

Thus, there remains a need for methods of imaging, diagnosing and treating brain tumors.

### SUMMARY OF THE INVENTION

A new target on brain tumor cells has now been identified and is useful in methods designed for the diagnosis and treatment of brain tumors. This target is the calcitonin receptor (CTR).

Accordingly, the present invention provides a method for the localisation, diagnosis, prognosis, and/or prediction of therapeutic outcome of a brain tumor in a subject, the method comprising detecting calcitonin receptor in brain cells of the subject, wherein the presence of calcitonin receptor localises, is diagnostic, prognostic and/or predictive for, the brain tumor.

In one embodiment, the method comprises administering to the subject a compound that binds calcitonin receptor, allowing the compound to bind to cells within the subject, and determining the location of the compound within the brain of the subject.

In another embodiment, the method comprises determining the location of a compound which binds calcitonin receptor in a subject, wherein the subject has been administered with the compound.

In another embodiment, the method comprises detecting calcitonin receptor in a sample obtained from the subject.

In an embodiment, the method comprises contacting the sample with a compound that binds calcitonin receptor.

Preferably, the compound is detectably labelled.

In yet another embodiment, the method comprises contacting the sample with a nucleic acid that hybridises with a polynucleotide encoding the calcitonin receptor.

In one particular embodiment, the polynucleotide is mRNA.

In another embodiment, the method comprises determining the level of calcitonin receptor in the brain cells of the subject and comparing the level of calcitonin receptor in the brain cells of the subject with a control, wherein a higher level of calcitonin receptor compared to the control localises, is diagnostic, prognostic and/or predictive for, the brain tumor.

The skilled person will understand that when the method of the invention is used for imaging the brain of a patient, the control may comprise an area of a scan, for example an MRI scan, known to correspond to a region of normal brain tissue.

In another aspect, the present invention provides a method of treatment comprising:

(i) performing the method of localisation, diagnosis, prognosis and/or prediction according to the invention; and

(ii) administering or recommending a therapeutic for the treatment of the brain tumor.

In yet another aspect, the present invention provides a method for treating or preventing a brain tumor in a subject, the method comprising administering to the subject an effective amount of a compound that binds calcitonin receptor to inhibit the growth of, or kill, brain tumor cells in the subject.

In one embodiment, the compound is conjugated to a cytotoxic agent or biological response modifier.

Preferably, the cytotoxic agent is a toxin, a chemotherapeutic agent, or a radioactive agent.

In one embodiment, the biological response modifier is a lymphokine, a cytokine, interferon or growth factor.

The methods of treating or preventing brain tumors involving the use of compounds which bind calcitonin receptor may be performed in isolation or as an adjunct to other therapeutic regimes, including for example other chemotherapy or radiotherapy regimes. Thus, in an embodiment, the method for treating or preventing the brain tumor is performed in combination with, prior to and/or after treatment with, a chemotherapeutic, such as, for example, temozolide or carmustine, or a radiotherapeutic.

In one embodiment of the methods of the invention, the compound binds an epitope of calcitonin receptor and the epitope comprises an amino acid sequence selected from SEQ ID NOs:3, 4 and 5.

The compound that binds the calcitonin receptor may be, for example, any polypeptide, ligand or other molecule identified as having binding affinity to calcitonin receptor.

In one embodiment of the method of the invention, the compound comprises an antibody. The antibody may be, for example, a monoclonal antibody, a chimeric antibody or a humanised antibody.

In one embodiment, the antibody is selected from 9B4 and 1C11 as described in WO 2009/039584.

The present inventors have found that administering a calcitonin receptor agonist to cells expressing calcitonin receptor alters the cell cycle and reduces proliferation of the cells.

Thus, in one embodiment the method comprises administering to the subject a calcitonin receptor agonist.

In one embodiment, the calcitonin receptor agonist is calcitonin or a calcitonin binding analogue.

In another embodiment, the calcitonin receptor agonist is an antibody.

In yet another embodiment, the calcitonin receptor agonist reduces proliferation of brain tumor cells.

In another embodiment, the calcitonin receptor agonist reduces tumor expansion.

In one embodiment, wherein the brain tumor is a glioma.

In one particular embodiment, the glioma is glioblastoma multiforme.

In another embodiment, the method of treatment is performed in combination with, prior to and/or after treatment with a chemotherapeutic or radiotherapeutic.

In another aspect, the present invention provides a method for treating or preventing a brain tumor in a subject, the method comprising administering to the subject an effective amount of a compound that reduces the production and/or activity of calcitonin receptor in brain cells of the subject.

In one embodiment, the compound reduces the level of calcitonin receptor mRNA in the brain cells.

In another embodiment, the method is performed in combination with, prior to and/or after treatment with a chemotherapeutic or radiotherapeutic.

Preferably, the compound is selected from an antisense polynucleotide, a catalytic polynucleotide, a microRNA and a dsRNA.

In one embodiment, the dsRNA is a siRNA or shRNA.

In one embodiment, the subject is a mammal, preferably a human.

In another aspect, the present invention provides a method of screening for a compound for the treatment of a brain tumor, the method comprising the steps of:

i) contacting calcitonin receptor or an epitope thereof with one or more candidate compounds,

ii) identifying a candidate compound which binds to the calcitonin receptor; and

iii) determining whether the compound inhibits the activity or division of, or kills, calcitonin receptor expressing brain tumor cells.

In a preferred embodiment of the method of the invention, the brain cells are malignant glial cells.

In yet another aspect, the present invention provides use of a compound that binds calcitonin receptor for the manufacture of a medicament for the treatment or prevention of a brain tumor.

In another aspect, the present invention provides use of a compound that binds calcitonin receptor for the treatment or prevention of a brain tumor.

In another aspect, the present invention provides use of a calcitonin receptor agonist for the manufacture of a medicament for the treatment or prevention of a brain tumor.

In one aspect, the present invention provides use of calcitonin receptor agonist in the treatment or prevention of a brain tumor.

In another aspect, the present invention provides use of a compound that reduces the production and/or activity of calcitonin receptor for the manufacture of a medicament for the treatment or prevention of a brain tumor.

In yet another aspect, the present invention provides use of a compound that reduces the production and/or activity of calcitonin receptor for the treatment or prevention of a brain tumor.

In another aspect, the present invention provides use of a compound that binds calcitonin receptor for the manufacture of a composition for the localisation of calcitonin receptor expressing brain tumor cells.

In another aspect, the present invention provides use of a compound that binds calcitonin receptor for the localisation of calcitonin receptor expressing brain tumor cells.

In one embodiment, the brain tumor is a glioma, for example glioblastoma multiforme.

In another aspect, the present invention provides a method of sensitizing tumor cells in a subject to chemotherapy, the method comprising administering to the subject a compound that reduces the production and/or activity of calcitonin receptor in tumor cells of the subject, and/or that reduces binding of calcitonin to calcitonin receptor in tumor cells of the subject.

In one embodiment, the method further comprises administering a chemotherapeutic to the subject. For example, the method may be performed in combination with and/or prior to treatment with a chemotherapeutic or radiotherapeutic.

In this aspect of the invention, the tumor cells may be cells of any solid tumor. Examples of solid tumors include adrenocarcinoma, brain tumor, breast, cervical, colorectal, endometrial, prostrate, gastric, liver, lung, lymphomas, melanoma, neuroblastoma, osteogenic sarcoma, ovarian, retinoblastoma, soft tissue sarcomas, testicular, as well as other tumors which respond to chemotherapy.

In one embodiment the tumor cells are brain tumor cells.

In an embodiment, the brain tumor is a glioma. In one particular embodiment, the glioma is glioblastoma multiforme.

In yet another embodiment, the tumor is not breast cancer.

The compound may be, for example, any polypeptide, ligand or other molecule which reduces the production and/or activity of calcitonin receptor in tumor cells of the subject, and/or that reduces binding of calcitonin to calcitonin receptor in tumor cells of the subject.

In one embodiment, the compound is a nucleic acid that reduces the production and/or activity of calcitonin receptor.

Alternatively, the compound may be an antibody that binds calcitonin receptor and reduces binding of calcitonin to the receptor.

As will be apparent, preferred features and characteristics of one aspect of the invention are applicable to many other aspects of the invention.

Throughout this specification the word “comprise”, or variations such as “comprises” or “comprising”, will be understood to imply the inclusion of a stated element, integer or step, or group of elements, integers or steps, but not the exclusion of any other element, integer or step, or group of elements, integers or steps.

The invention is hereinafter described by way of the following non-limiting Examples and with reference to the accompanying figures.

### BRIEF DESCRIPTION OF THE ACCOMPANYING DRAWINGS

FIG. 1. A. Lanes 1 to 8 represent immunoblots with membrane preparations (total membrane prep., lanes 1-4 and enriched plasma membrane prep. Lanes 5-8). Lanes 1 and 2 represent proteins probed with anti-CTR antibody, MCA2191, and Lanes 3 and 4 with anti-CTR antibody, 9B4 from COS-7/CTR-positive (Lanes 1 and 3), COS-7/CTR-negative (Lanes 2 and 4) stably transfected cell lines. The amounts of total membrane protein loaded onto each lane were 25 μg (lanes 1-4). Plasma membrane preps. from 3T3 stable transfected cell lines were loaded onto lanes 5-8 (60 μg each). Lanes 5 and 7 represent proteins from parental flpIN 3T3 cells (CTR-negative). Lanes 6 and 8 represent proteins prepared from flpIN 3T3 stable transfected cell line using the flpIN system to express cMyc tagged hCTR.

The blots developed with anti-CTR antibodies MCA 2191 (Lanes 1-2 and 5-6) and 9B4 (Lanes 3-4 and 7-8). Primary anti-CTR antibodies were used at a concentration of 10 μg/mL (Lanes 1-4), and 1 μg/mL (Lanes 5-8). For development in Lanes 1-4 the Pierce ECL system was used and the concentration of the secondary goat anti-mouse HRP was 1 μg/mL. For development in Lanes 5-8 the secondary, goat anti-mouse AF-647 (Molecular Probes, USA) was used at 0.5 μg/mL. Specific bands for fully glycosylated (Band A) and unglycosylated (Band B) hCTR are indicated. BioRad Precision molecular weight standards were used to indicate apparent molecular weights in kiloDaltons (kD).

B. Confocal images of cultured cell lines COS-7/CTR-positive (panels A-C) and COS-7/CTR-negative (panels D-F), are shown in which a series of Z-plane stacked images have been compressed into each single image (devolution). The staining identified at 488 nm corresponds to the anti-CTR antibodies 9B4 (panels A and D), MCA2191 (panels B and E), and the IgG2A isotype control (panels C and F). The staining of nuclei with DAPI was captured at 405 nm. Images were captured using a X20 objective lens, overall magnification X200. The calibration bar line in panel F represents 60 μm for each panel.

FIG. 2. Glioblastoma multiforme (GBM) tissue fixed in buffered formalin from patient #2. Panels A, C and E are from similar fields in adjacent sections. The boxed areas in each of these panels correspond to the areas of higher magnification shown panels B, D and F, respectively. Panels A and B are images from sections counter-stained with haematoxylin and eosin and were captured using the objective magnifications Obj.×20 and Obj.×100 respectively. In panel B, GB cells (arrows) have a large pink cytoplasm and often irregular pale nucleus with chromatin condensations and a prominent nucleolus. Some of the GB cells are multi-nucleate (arrow head). In panels C and D, stained using the anti-hCTR antibody MCA 2191 (dilution 1:3000), show CTR-ir confined to cell bodies and processes. Note that the CTR-ir cells have large pale nuclei with condensations of chromatin and often a prominent nucleolus (examples are indicated with arrows). Some of these CTR-ir cells are multi-nucleate (arrow heads). There are many examples of CTR-negative cells (examples are indicated with *). In panels E and F tissue, stained using the anti-hCTR antibody 9B4 (dilution 1:400), show CTR-ir also confined to cell bodies and processes, with characteristics as described for panel D. The scale bar in panel F equals 170 μm in A, B and C, and 18 μm in B, D and F.

FIG. 3. Immunohistochemical analysis of thin sections from the tumour from patient #4. The tissue was fixed in 4% paraformaldehyde/PBS. In panel A is shown at low magnification (Obj.×20) a region of the tumour stained with the anti-hCTR antibody (MCA 2191, diluted 1:2000). In panels B (box in A) and D (box in B) the images were captured at higher magnifications, Obj.×40 and Obj.×100 respectively. In panel C a neighbouring section was stained with an anti-hCLR antibody (AB 9414, diluted 1:500), magnification Obj.×40. The insert in panel C shows small vessels with CLR-positive cells. In panels E & F similar regions in neighbouring sections were stained with the anti-GFAP antibody (MAB 3402, diluted 1:3000) using magnifications Obj.×40 and Obj.×100, respectively. In panels G & H are shown images of staining with anti-hCTR antibody MCA2191 (diluted 1:3000) and pre-neutralised with 100-fold molar excess of the antigenic peptide, respectively, using magnifications of Obj.×40. The scale bar in panel H equals 85 μm in A, 50 μm in B, C and E, 40 μm in G and H, and 18 μm in D and F.

FIG. 4. Immunohistochemical analysis of thin sections from four different human tumours, glioblastoma multiforme with tissue fixed in buffered formalin. In all panels the anti-hCTR antibody MCA 2191 was used at a dilution of 1:4000. In panel A is shown at low magnification (Obj.×20) a region of one tumour, and at higher magnification (Obj.×100) in panel B. In panel C & D are images from a second patient (magnification Obj.×20 and Obj.×100). In panels E & F are regions of brain tumour from a third patient (magnifications Obj.×10 and Obj.×100). In panel G is shown a region of normal brain that was situated adjacent to a tumour and stained with the anti-hCTR antibody (magnification Obj.×40). In panel H CTR-ir cells from a fourth patient showed more ordered alignment of cells (magnification Obj.×100). The scale bar in panel H equals 170 μm in E, 85 μm in A and C, 42 μm in G, and 18 μm in B, D, F and H.

FIG. 5. Co-localisation of markers determined with multi-labelled immuno-fluorescence in confocal images of fixed/frozen GBM patient samples (patient #13, A-D; patient #14, panels E-L. In A-D is a confocal image of tissue from patient #13 stained with anti-CTR (MCA2191, panel B, red), anti-GFAP (panel C, green) and anti-nestin (panel D, pink) antibodies. The composite is shown in panel A, in which the arrows indicate cells positive for CTR, GFAP and nestin while the arrowheads indicate cells positive only for CTR and nestin. Multiple images in the Z-plane were recorded and devolution performed to form a single image. In E-L are shown examples of confocal images of individual cells from patient #14 stained with anti-GFAP (panels F and J, red), CD133 (panels G and K, green) and anti-CTR (panels H and L, pink) antibodies. The composite is shown in panels E and I. Nuclei are stained with DAPI and detected at 405 nm (blue).

FIG. 6. Inhibition of phosphorylation of ERK1/2 (A) and stimulation of cAMP production (B) in the GBM cell line A172 with the agonist human CT (hCT, in A & B) and inhibition with inverse agonist of CTR, 10⁻⁶ M sCT (8-32) [▾ in A, ▪ in B]. Log EC₅₀: (A) −10.5 and (B) −9.9. (C) is an immunoblot of a crude membrane preparation from the cell line A172 and probed with the anti-CTR antibody MCA2191.

### KEY TO THE SEQUENCE LISTING

SEQ ID NO:1—Nucleotide sequence of human CTR
SEQ ID NO:2—Amino acid sequence of human CTR
SEQ ID NO:3—Epitope 1 of human CTR
SEQ ID NO:4—Epitope 4 of human CTR
SEQ ID NO:5—Epitope 5 of human CTR

### DETAILED DESCRIPTION OF THE INVENTION

Unless specifically defined otherwise, all technical and scientific terms used herein shall be taken to have the same meaning as commonly understood by one of ordinary skill in the art (e.g., in protein chemistry, biochemistry, cell culture, molecular genetics, microbiology, immunology and immunohistochemistry).

Unless otherwise indicated, the recombinant protein, cell culture, and immunological techniques utilized in the present invention are standard procedures, well known to those skilled in the art. Such techniques are described and explained throughout the literature in sources such as, J. Perbal, A Practical Guide to Molecular Cloning, John Wiley and Sons (1984), J. Sambrook et al., Molecular Cloning: A Laboratory Manual, 3^{rd} edn, Cold Spring Harbour Laboratory Press (2001), T. A. Brown (editor), Essential Molecular Biology: A Practical Approach, Volumes 1 and 2, IRL Press (1991), D. M. Glover and B. D. Hames (editors), DNA Cloning: A Practical Approach, Volumes 1-4, IRL Press (1995 and 1996), and F. M. Ausubel et al. (editors), Current Protocols in Molecular Biology, Greene Pub. Associates and Wiley-Interscience (1988, including all updates until present), Ed Harlow and David Lane (editors) Antibodies: A Laboratory Manual, Cold Spring Harbour Laboratory, (1988), and J. E. Coligan et al. (editors) Current Protocols in Immunology, John Wiley & Sons (including all updates until present).

As used herein, the term “subject” refers to an animal such as a mammal, e.g. humans or non-human mammals such as cats, dogs, cattle, sheep, horses, rabbits and monkeys. In a preferred embodiment, the subject is a human.

The “sample” may be of any suitable type and may refer, e.g., to a material suspected of containing calcitonin receptor expressing cells. The sample can be used as obtained directly from the source or following at least one step of (partial) purification. The sample can be prepared in any convenient medium which does not interfere with the method of the invention. Typically, the sample is an aqueous solution, biological fluid, cells or tissue. The sample can be used as obtained directly from the source or following at least one step of (partial) purification. Pre-treatment may involve, for example, diluting viscous fluids, and the like. Treatment of a sample can involve filtration, distillation, separation, concentration, inactivation of interfering components, and the addition of reagents. The selection and pre-treatment of biological samples prior to testing is well known in the art and need not be described further.

As used herein “brain tumor” refers to an abnormal growth of cells in the brain, and in particular refers to malignant tumors, or brain cancers. Examples of brain tumors include astrocytoma (including low-grade glioma, high-grade astrocytoma, and glioblastoma), meningioma, oligodendroglioma, medulloblastoma, ependymoma, brain stem glioma and glioblastoma multiforme.

As used herein, the term “epitope” refers to a region of calcitonin receptor as described herein which may be bound by an antibody.

As used herein “binds an epitope” means that an antibody need only bind within the given amino acid sequence, and need not bind the entire amino acid sequence.

“Administering” as used herein is to be construed broadly and includes administering a compound as described herein to a subject as well as providing a compound as described herein to a cell.

As used herein, “detectably labelled” refers to a compound which is labelled with a moiety that is detectable by spectroscopic, photochemical, biochemical, immunochemical, chemical, or other physical means. Non-limiting examples of useful detectable labels include ³²P, fluorescent dyes, electron-dense reagents, enzymes (e.g., as commonly used in an ELISA), biotin, digoxigenin, or proteins which can be made detectable, e.g., by incorporating a radiolabel into the protein.

As used herein, the terms “treating”, “treat” or “treatment” include administering a therapeutically effective amount of a compound as described herein sufficient to reduce or delay the onset or progression of a brain tumor, or to reduce or eliminate at least one symptom of the brain tumor.

As used herein, the terms “preventing”, “prevent” or “prevention” include administering a therapeutically effective amount of a compound useful for the invention sufficient to stop or hinder the development of at least one symptom of the brain tumor.

As used herein, the term “diagnosis”, and variants thereof such as, but not limited to, “diagnose”, “diagnosed” or “diagnosing” includes any primary diagnosis of a clinical state or diagnosis of recurrent disease.

“Prognosis”, “prognosing” and variants thereof as used herein refer to the likely outcome or course of a disease.

As used herein, the phrase “prediction of therapeutic outcome” and the terms “predicting”, “predictive” and variants thereof refer to determining the probability of response to a therapeutic agent or modality, for example, determining the probability of the sensitivity of a brain tumor cell to a chemotherapeutic agent or a radiotherapeutic agent, or determining the probability of survival or recurrence of disease.

By “reduces” or “reducing” the production or activity of calcitonin receptor in a cell is meant a decrease in the production or activity of calcitonin receptor in a cell in the presence of a compound when compared to the production or activity of calcitonin receptor in the cell in the absence of the compound, such as in a control sample. The degree of decrease in the production or activity of calcitonin receptor will vary with the nature and quantity of the compound present, but will be evident e.g., as a detectable decrease in the production or activity of calcitonin receptor; desirably a degree of decrease greater than 5%, 10%, 33%, 50%, 75%, 90%, 95% or 99% as compared to the production or activity of calcitonin receptor in the absence of the compound.

### Calcitonin Receptor

The calcitonin receptor belongs to the type II seven transmembrane domain G-protein-coupled receptors. Porcine calcitonin receptor was the first to be cloned (Lin et al., 1991). Shortly afterwards, the human, and several other species, of calcitonin receptor were cloned and characterised (Goldring et al. 1993). The nucleotide sequence of human calcitonin receptor is provided as SEQ ID NO:1, and the amino acid sequence is provided as SEQ ID NO:2. The physiological function of the thyrocalcitonin (CT)/receptor (CTR) complex has been previously described in terms of a homeostatic mechanism for calcium, which was active under conditions of hypercalcaemia (Copp et al., 1962; Hirsch and Baruch, 2003; Hirsch et al., 1964).

Reference to calcitonin receptor as used herein includes isoforms, splice variants and allelic variants of calcitonin receptor as would be understood by one skilled in the art (see, for example, Gorn et al., 1995; Nakamura et al., 1997; Masi et al., 1998).

Compounds that Bind Calcitonin Receptor

The present inventors have now shown that the calcitonin receptor (CTR) is expressed by cells associated with brain tumors. Thus, compounds that bind to CTR will be useful for the localisation, diagnosis, prognosis, and/or prediction of therapeutic outcome of brain tumors. In addition, antibodies directed against CTR will be capable of killing brain tumor cells through mechanisms such as antibody-dependent cell-mediated cytotoxicity (ADCC), complement dependent cytotoxicity (CDC) and apoptosis and will therefore be effective therapeutic agents brain tumor cells. Compounds directed against CTR can also be used to deliver cytotoxins to brain tumor cells.

Compounds that bind CTR that are useful in the present invention may be any compound, e.g. a polypeptide, ligand or other molecule, identified as having binding affinity to CTR. The binding between a compound and CTR may be mediated by covalent or non-covalent interactions or a combination of covalent and non-covalent interactions. When the interaction of the compound and CTR produces a non-covalently bound complex, the binding which occurs is typically electrostatic, hydrogen-bonding, or the result of hydrophilic/lipophilic interactions. Particularly preferred compounds that bind CTR are anti-CTR antibodies.

Although not essential, the compound may bind specifically to CTR. The phrase “bind specifically,” means that under particular conditions, the compound binds CTR and does not bind to a significant amount to other, for example, proteins or carbohydrates. Specific binding to CTR under such conditions may require an antibody that is selected for its specificity. A variety of immunoassay formats may be used to select antibodies specifically immunoreactive with CTR. For example, solid-phase ELISA immunoassays are routinely used to select antibodies specifically immunoreactive with a protein or carbohydrate. See Harlow and Lane (1988) Antibodies, a Laboratory Manual, Cold Spring Harbor Publications, New York, for a description of immunoassay formats and conditions that can be used to determine specific immunoreactivity.

### Antibodies

The term “antibody” as used herein includes polyclonal antibodies, monoclonal antibodies, bispecific antibodies, diabodies, triabodies, heteroconjugate antibodies, chimeric antibodies, humanised antibodies including intact molecules as well as fragments thereof, and other antibody-like molecules. Antibodies include modifications in a variety of forms including, for example, but not limited to, domain antibodies including either the VH or VL domain, a dimer of the heavy chain variable region (VHH, as described for a camelid), a dimer of the light chain variable region (VLL), Fv fragments containing only the light (VL) and heavy chain (VH) variable regions which may be joined directly or through a linker, or Fd fragments containing the heavy chain variable region and the CH1 domain. A scFv consisting of the variable regions of the heavy and light chains linked together to form a single-chain antibody (Bird et al., 1988; Huston et al., 1988) and oligomers of scFvs such as diabodies and triabodies are also encompassed by the term “antibody”. Also encompassed are fragments of antibodies such as Fab, (Fab′)2 and FabFc2 fragments which contain the variable regions and parts of the constant regions. Complementarity determining region (CDR)-grafted antibody fragments and oligomers of antibody fragments are also encompassed. The heavy and light chain components of an Fv may be derived from the same antibody or different antibodies thereby producing a chimeric Fv region. The antibody may be of animal (for example mouse, rabbit or rat) or human origin or may be chimeric (Morrison et al., 1984) or humanized (Jones et al., 1986). As used herein the term “antibody” includes these various forms. Using the guidelines provided herein and those methods well known to those skilled in the art which are described in the references cited above and in such publications as Harlow & Lane, Antibodies: a Laboratory Manual, Cold Spring Harbor Laboratory, (1988) the antibodies for use in the methods of the present invention can be readily made.

The antibodies may be Fv regions comprising a variable light (VL) and a variable heavy (VH) chain in which the light and heavy chains may be joined directly or through a linker. As used herein a linker refers to a molecule that is covalently linked to the light and heavy chain and provides enough spacing and flexibility between the two chains such that they are able to achieve a conformation in which they are capable of specifically binding the epitope to which they are directed. Protein linkers are particularly preferred as they may be expressed as an intrinsic component of the Ig portion of the fusion polypeptide.

In another embodiment, recombinantly produced single chain scFv antibody, preferably a humanized scFv, is used in the methods of the invention.

In one embodiment, the antibodies have the capacity for intracellular transmission. Antibodies which have the capacity for intracellular transmission include antibodies such as camelids and llama antibodies, shark antibodies (IgNARs), scFv antibodies, intrabodies or nanobodies, for example, scFv intrabodies and VHH intrabodies. Such antigen binding agents can be made as described by Harmsen and De Haard, 2007; Tibary et al., 2007; Muyldermans, 2001; and references cited therein.

Anti-CTR antibodies will be known to those skilled in the art and have been used to detect CTR expression in certain tissues. CTR has not been used to date, however, as a target for the treatment of brain tumors, or for the detection or localization of CTR expressing cell in brain tumors. Examples of suitable anti-CTR antibodies include the monoclonal antibodies 1C11 and 9B4 disclosed in WO 2009/039584, and MAB4614 (R&D Systems, Inc., USA) which recognizes a discontinuous epitope of CTR.

### Monoclonal Antibodies

Monoclonal antibodies directed against CTR epitopes can be readily produced by one skilled in the art. The general methodology for making monoclonal antibodies by hybridomas is well known. Immortal antibody-producing cell lines can be created by cell fusion, and also by other techniques such as direct transformation of B lymphocytes with oncogenic DNA, or transfection with Epstein-Barr virus. Panels of monoclonal antibodies produced against CTR epitopes can be screened for various properties; i.e. for isotype and epitope affinity.

Animal-derived monoclonal antibodies can be used for direct in vivo immunotherapy. However, it has been observed that when, for example, mouse-derived monoclonal antibodies are used in humans as therapeutic agents, the patient produces human anti-mouse antibodies. Thus, animal-derived monoclonal antibodies are not preferred for therapy, especially for long term use. With established genetic engineering techniques it is possible, however, to create chimeric or humanized antibodies that have animal-derived and human-derived portions. The animal can be, for example, a mouse or other rodent such as a rat.

If the variable region of the chimeric antibody is, for example, mouse-derived while the constant region is human-derived, the chimeric antibody will generally be less immunogenic than a “pure” mouse-derived monoclonal antibody. These chimeric antibodies would likely be more suited for therapeutic use, should it turn out that “pure” mouse-derived antibodies are unsuitable.

Methodologies for generating chimeric antibodies are available to those in the art. For example, the light and heavy chains can be expressed separately, using, for example, immunoglobulin light chain and immunoglobulin heavy chains in separate plasmids. These can then be purified and assembled in vitro into complete antibodies; methodologies for accomplishing such assembly have been described (see, for example, Sun et al., 1986). Such a DNA construct may comprise DNA encoding functionally rearranged genes for the variable region of a light or heavy chain of an anti-CTR antibody linked to DNA encoding a human constant region. Lymphoid cells such as myelomas or hybridomas transfected with the DNA constructs for light and heavy chain can express and assemble the antibody chains.

In vitro reaction parameters for the formation of IgG antibodies from reduced isolated light and heavy chains have also been described (see, for example, Beychok, 1979). Co-expression of light and heavy chains in the same cells to achieve intracellular association and linkage of heavy and light chains into complete H2L2 IgG antibodies is also possible. Such co-expression can be accomplished using either the same or different plasmids in the same host cell.

### Humanising Methodologies/Techniques

In another preferred embodiment of the present invention the anti-CTR antibody is humanized, that is, an antibody produced by molecular modelling techniques wherein the human content of the antibody is maximised while causing little or no loss of binding affinity attributable to the variable region of, for example, a parental rat, rabbit or murine antibody.

An antibody may be humanized by grafting the desired CDRs onto a human framework according to EP-A-0239400. A DNA sequence encoding the desired reshaped antibody can therefore be made beginning with the human DNA whose CDRs it is wished to reshape. The animal-derived variable domain amino acid sequence containing the desired CDRs is compared to that of the chosen human antibody variable domain sequence. The residues in the human variable domain are marked that need to be changed to the corresponding residue in the animal to make the human variable region incorporate the animal-derived CDRs. There may also be residues that need substituting in, adding to or deleting from the human sequence.

Oligonucleotides are synthesized that can be used to mutagenize the human variable domain framework to contain the desired residues. Those oligonucleotides can be of any convenient size. The method of oligonucleotide-directed in vitro mutagenesis is well known.

Alternatively, humanisation may be achieved using the recombinant polymerase chain reaction (PCR) methodology of WO 92/07075. Using this methodology, a CDR may be spliced between the framework regions of a human antibody. In general, the technique of WO 92/07075 can be performed using a template comprising two human framework regions, AB and CD, and between them, the CDR which is to be replaced by a donor CDR. Primers A and B are used to amplify the framework region AB, and primers C and D used to amplify the framework region CD. However, the primers B and C each also contain, at their 5′ ends, an additional sequence corresponding to all or at least part of the donor CDR sequence. Primers B and C overlap by a length sufficient to permit annealing of their 5′ ends to each other under conditions which allow a PCR to be performed. Thus, the amplified regions AB and CD may undergo gene splicing by overlap extension to produce the humanized product in a single reaction.

Following the mutagenesis reactions to reshape the antibody, the mutagenised DNAs can be linked to an appropriate DNA encoding a light or heavy chain constant region, cloned into an expression vector, and transfected into host cells, preferably mammalian cells. These steps can be carried out in routine fashion. A reshaped antibody may therefore be prepared by a process comprising:

(a) preparing a first replicable expression vector including a suitable promoter operably linked to a DNA sequence which encodes at least a variable domain of an Ig heavy or light chain, the variable domain comprising framework regions from a human antibody and the CDRs required for the humanized antibody of the invention;

(b) preparing a second replicable expression vector including a suitable promoter operably linked to a DNA sequence which encodes at least the variable domain of a complementary Ig light or heavy chain respectively;

(c) transforming a cell line with the first or both prepared vectors; and

(d) culturing said transformed cell line to produce said altered antibody.

Preferably the DNA sequence in step (a) encodes both the variable domain and each constant domain of the human antibody chain. The humanized antibody can be prepared using any suitable recombinant expression system. The cell line which is transformed to produce the altered antibody may be a Chinese Hamster Ovary (CHO) cell line or an immortalised mammalian cell line, which is advantageously of lymphoid origin, such as a myeloma, hybridoma, trioma or quadroma cell line. The cell line may also comprise a normal lymphoid cell, such as a B-cell, which has been immortalised by transformation with a virus, such as the Epstein-Barr virus. Most preferably, the immortalised cell line is a myeloma cell line or a derivative thereof.

The CHO cells used for expression of the antibodies may be dihydrofolate reductase (dhfr) deficient and so dependent on thymidine and hypoxanthine for growth. The parental dhfr⁻ CHO cell line is transfected with the DNA encoding the antibody and dhfr gene which enables selection of CHO cell transformants of dhfr positive phenotype. Selection is carried out by culturing the colonies on media devoid of thymidine and hypoxanthine, the absence of which prevents untransformed cells from growing and transformed cells from resalvaging the folate pathway and thus bypassing the selection system. These transformants usually express low levels of the DNA of interest by virtue of co-integration of transfected DNA of interest and DNA encoding dhfr. The expression levels of the DNA encoding the antibody may be increased by amplification using methotrexate (MTX). This drug is a direct inhibitor of the enzyme dhfr and allows isolation of resistant colonies which amplify their dhfr gene copy number sufficiently to survive under these conditions. Since the DNA sequences encoding dhfr and the antibody are closely linked in the original transformants, there is usually concomitant amplification, and therefore increased expression of the desired antibody.

Another preferred expression system for use with CHO or myeloma cells is the glutamine synthetase (GS) amplification system described in WO 87/04462. This system involves the transfection of a cell with DNA encoding the enzyme GS and with DNA encoding the desired antibody. Cells are then selected which grow in glutamine free medium and can thus be assumed to have integrated the DNA encoding GS. These selected clones are then subjected to inhibition of the enzyme GS using methionine sulphoximine (Msx). The cells, in order to survive, will amplify the DNA encoding GS with concomitant amplification of the DNA encoding the antibody.

Although the cell line used to produce the humanized antibody is preferably a mammalian cell line, any other suitable cell line, such as a bacterial cell line or a yeast cell line, may alternatively be used. In particular, it is envisaged that E. coli-derived bacterial strains could be used.

Once expressed, the whole antibodies, their dimers, individual light and heavy chains, or other immunoglobulin forms can be recovered and purified according to standard procedures of the art, including ammonium sulfate precipitation, affinity columns, column chromatography, gel electrophoresis and the like (See, generally, Scopes, R., Protein Purification, 3^{rd} ed., Springer-Verlag, N.Y. (1994)). Substantially pure immunoglobulins of at least about 90 to 95% homogeneity are preferred, and 98 to 99% or more homogeneity most preferred, for pharmaceutical uses. Once purified, partially or to homogeneity as desired, a humanized antibody may then be used therapeutically or in developing and performing assay procedures, immunofluorescent stainings, and the like.

Studies carried out by Greenwood et al. (1993) have demonstrated that recognition of the Fc region of an antibody by human effector cells can be optimised by engineering the constant region of the immunoglobulin molecule. This could be achieved by fusing the variable region genes of the antibody, with the desired specificity, to human constant region genes encoding immunoglobulin isotypes that have demonstrated effective ADCC in human subjects, for example the IgG1 and IgG3 isotypes (Greenwood and Clark (1993) Protein Engineering of Antibody Molecules for Prophylactic and Therapeutic Applications in Man. Edited by Mike Clark, published by Academic Titles. Section II 85-113). The resulting chimeric or humanized antibodies to CTR should be particularly effective in inducing ADCC.

Antibodies with fully human variable regions against CTR can also be prepared by administering the antigen to a transgenic animal which has been modified to produce such antibodies in response to antigenic challenge, but whose endogenous loci have been disabled. Various subsequent manipulations can be performed to obtain either antibodies per se or analogs thereof (see, for example, U.S. Pat. No. 6,075,181).

### Preparation of Genes Encoding Antibodies or Fragments Thereof

Genes encoding antibodies, both light and heavy chain genes or portions thereof, e.g., single chain Fv regions, may be cloned from a hybridoma cell line. They may all be cloned using the same general strategy. Typically, for example, poly(A)⁺mRNA extracted from the hybridoma cells is reverse transcribed using random hexamers as primers. For Fv regions, the V_{H} and V_{L} domains are amplified separately by two polymerase chain reactions (PCR). Heavy chain sequences may be amplified using 5′ end primers which are designed according to the amino-terminal protein sequences of the anti-CTR heavy chains respectively and 3′ end primers according to consensus immunoglobulin constant region sequences (Kabat et al., Sequences of Proteins of Immunological Interest. 5th edition. U.S. Department of Health and Human Services, Public Health Service, National Institutes of Health, Bethesda, Md. (1991)). Light chain Fv regions are amplified using 5′ end primers designed according to the amino-terminal protein sequences of anti-CTR light chains and in combination with the primer C-kappa. One of skill in the art would recognize that many suitable primers may be employed to obtain Fv regions.

The PCR products are subcloned into a suitable cloning vector. Clones containing the correct size insert by DNA restriction are identified. The nucleotide sequence of the heavy or light chain coding regions may then be determined from double stranded plasmid DNA using sequencing primers adjacent to the cloning site. Commercially available kits may be used to facilitate sequencing the DNA. DNA encoding the Fv regions may be prepared by any suitable method, including, for example, amplification techniques such as PCR and LCR.

Chemical synthesis produces a single stranded oligonucleotide. This may be converted into double stranded DNA by hybridization with a complementary sequence, or by polymerization with a DNA polymerase using the single strand as a template. While it is possible to chemically synthesize an entire single chain Fv region, it is preferable to synthesize a number of shorter sequences (about 100 to 150 bases) that are later ligated together.

Alternatively, sub-sequences may be cloned and the appropriate subsequences cleaved using appropriate restriction enzymes. The fragments may then be ligated to produce the desired DNA sequence.

Once the Fv variable light and heavy chain DNA is obtained, the sequences may be ligated together, either directly or through a DNA sequence encoding a peptide linker, using techniques well known to those of skill in the art. In one embodiment, heavy and light chain regions are connected by a flexible peptide linker (e.g., (Gly₄Ser)₃) which starts at the carboxyl end of the heavy chain Fv domain and ends at the amino terminus of the light chain Fv domain. The entire sequence encodes the Fv domain in the form of a single-chain antigen binding protein.

### Detecting Brain Tumors

In one embodiment, the present invention provides a method for the localisation, diagnosis, prognosis and/or prediction of therapeutic outcome of a brain tumor in a subject, the method comprising detecting the presence of calcitonin receptor in brain cells of a subject, wherein the presence of calcitonin receptor localises, is diagnostic, prognostic and/or predictive for the brain tumor.

### Protein Detection Techniques

In one embodiment, calcitonin receptor is detected in brain cells of a subject, wherein the detection of calcitonin receptor in the brain cells localises, is diagnostic, prognostic and/or predictive for the brain tumor. The method may comprise administering to the subject a compound that binds calcitonin receptor, allowing the compound to bind to cells within the subject, and determining the location of the compound within the brain of the subject. Alternatively, the method comprises contacting a biological sample derived from the subject with, for example, an antibody capable of binding to calcitonin receptor and detecting the formation of an antigen-antibody complex.

Preferred detection systems contemplated herein include any known assay for detecting proteins in a biological sample obtained from a subject, such as, for example, SDS/PAGE, isoelectric focussing, 2-dimensional gel electrophoresis comprising SDS/PAGE and isoelectric focussing, an immunoassay, flow cytometry e.g. fluorescence-activated cell sorting (FACS), a detection based system using an antibody or non-antibody compound, such as, for example, a small molecule (e.g. a chemical compound, agonist, antagonist, allosteric modulator, competitive inhibitor, or non-competitive inhibitor, of the protein). In accordance with these embodiments, the antibody or small molecule may be used in any standard solid phase or solution phase assay format amenable to the detection of proteins. Optical or fluorescent detection, such as, for example, using mass spectrometry, MALDI-TOF, biosensor technology, evanescent fiber optics, or fluorescence resonance energy transfer, is clearly encompassed by the present invention. Assay systems suitable for use in high throughput screening of mass samples, e.g. a high throughput spectroscopy resonance method (e.g. MALDI-TOF, electrospray MS or nano-electrospray MS), are also contemplated.

Immunoassay formats are particularly suitable, e.g., selected from the group consisting of, an immunoblot, a Western blot, a dot blot, an enzyme linked immunosorbent assay (ELISA), radioimmunoassay (RIA), enzyme immunoassay. Modified immunoassays utilizing fluorescence resonance energy transfer (FRET), isotope-coded affinity tags (ICAT), matrix-assisted laser desorption/ionization time of flight (MALDI-TOF), electrospray ionization (ESI), biosensor technology, evanescent fiber-optics technology or protein chip technology are also useful.

Preferably, the assay is a semi-quantitative assay or quantitative assay.

Standard solid phase ELISA formats are particularly useful in determining the concentration of a protein or antibody from a variety of patient samples.

In one form, such an assay involves immobilising a biological sample comprising antibodies against CTR or an immunogenic fragment thereof, onto a solid matrix, such as, for example a polystyrene or polycarbonate microwell or dipstick, a membrane, or a glass support (e.g. a glass slide).

In the case of an antigen-based assay, an antibody that specifically binds CTR is brought into direct contact with the immobilised biological sample, and forms a direct bond with any of its target protein present in said sample. For an antibody-based assay, an immobilized immunogenic fragment or epitope of CTR is contacted with the sample. The added antibody or protein in solution is generally labelled with a detectable reporter molecule, such as for example, a fluorescent label (e.g. Alexa Fluor® Dyes (Invitrogen), FITC or Texas Red) or an enzyme (e.g. horseradish peroxidase (HRP)), alkaline phosphatase (AP) or β-galactosidase. Alternatively, or in addition, a second labelled antibody can be used that binds to the first antibody or to the isolated/recombinant antigen. Following washing to remove any unbound antibody or antigen, as appropriate, the label is detected either directly, in the case of a fluorescent label, or through the addition of a substrate, such as for example hydrogen peroxide, TMB, or toluidine, or 5-bromo-4-chloro-3-indol-beta-D-galaotopyranoside (x-gal).

Such ELISA based systems are particularly suitable for quantification of the amount of a protein or antibody in a sample, such as, for example, by calibrating the detection system against known amounts of a standard.

In another form, an ELISA consists of immobilizing an antibody that specifically binds CTR on a solid matrix, such as, for example, a membrane, a polystyrene or polycarbonate microwell, a polystyrene or polycarbonate dipstick or a glass support. A patient sample is then brought into physical relation with said antibody, and the antigen in the sample is bound or ‘captured’. The bound protein can then be detected using a labelled antibody. For example if the protein is captured from a human sample, an anti-human antibody is used to detect the captured protein. Alternatively, a third labelled antibody can be used that binds the second (detecting) antibody.

### Nucleic Acid Detection Techniques

Any suitable technique that allows for the qualitative and/or quantitative assessment of the level of expression of the calcitonin receptor gene in a tissue may be used. Comparison may be made by reference to a standard control, or to a control level that is found in healthy tissue. For example, levels of a transcribed gene can be determined by Northern blotting, and/or RT-PCR. With the advent of quantitative (real-time) PCR, quantitative analysis of gene expression can be achieved by using appropriate primers for the gene of interest. The nucleic acid may be labelled and hybridised on a gene array, in which case the gene concentration will be directly proportional to the intensity of the radioactive or fluorescent signal generated in the array.

In one particular example, a brain tumor may be localised, diagnosed, prognosed or predicted by contacting nucleic acid in a sample obtained from a subject with a nucleic acid probe under stringent hybridisation conditions that allow the formation of a hybrid complex between the nucleic acid probe and the nucleotide sequence encoding CTR (SEQ ID NO: 1) and detecting the presence of a hybrid complex in the samples. It may be preferable to label the nucleic acid probe to aid its detection. This level of detection is compared to control levels, such as, for example, gene levels from a healthy specimen or a standard control; detection of altered levels of the hybrid complex from the patient tissue is indicative of a brain tumor.

The term “hybridization” or variants thereof as used here refers to the association of two nucleic acid molecules with one another by hydrogen bonding. Factors that affect this bonding include: the type and volume of solvent; reaction temperature; time of hybridization; agitation; agents to block the non-specific attachment of the liquid phase molecule to the solid support (Denhardt's reagent or BLOTTO); the concentration of the molecules; use of compounds to increase the rate of association of molecules (dextran sulphate or polyethylene glycol); and the stringency of the washing conditions following hybridization (see Sambrook et al. Molecular Cloning; A Laboratory Manual, Second Edition (1989)). In accordance with these principles, the inhibition of hybridization of a complementary molecule to a target molecule may be examined using a hybridization assay; a substantially homologous molecule possessing a greater degree of homology will then compete for and inhibit the binding of a completely homologous molecule to the target molecule under various conditions of stringency, as taught in Wahl and Berger (1987) and Kimmel (1987).

“Stringency” refers to conditions in a hybridization reaction that favour the association of very similar molecules over association of molecules that differ. High stringency hybridisation conditions are defined as overnight incubation at 42° C. in a solution comprising 50% formamide, 5×SSC (150 mM NaCl, 15 mM trisodium citrate, pH8.0), 50 mM sodium phosphate (pH7.6), 5×Denhardt's solution, 10% dextran sulphate, and 20 microgram/ml denatured, sheared salmon sperm DNA, followed by washing the filters in 0.1×SSC at approximately 65° C. Low stringency conditions involve the hybridisation reaction being carried out at 35° C. Preferably, the conditions used for hybridization in the methods of the present invention are those of high stringency.

The nucleic acid may be separated from the sample for testing. Suitable methods will be known to those of skill in the art. For example, RNA may be isolated from a cell sample to be analysed using conventional procedures, such as are supplied by QIAGEN technology. This RNA is then reverse-transcribed into DNA using reverse transcriptase and the DNA molecule of interest may then be amplified by PCR techniques using specific primers.

Diagnostic procedures may also be performed directly upon patient samples. Hybridisation or amplification assays, such as, for example, Southern or Northern blot analysis, immunohistochemistry, single-stranded conformational polymorphism analysis (SSCP) and PCR analyses are among techniques that are useful in this respect. If desired, target or probe nucleic acid may be immobilised to a solid support such as a microtitre plate, membrane, polystyrene bead, glass slide or other solid phase.

### Medical Imaging

Compounds that bind calcitonin receptor can be used in methods of imaging brain tumors. In particular, compounds that bind calcitonin receptor and which are conjugated or bound to, and/or coated with, a detectable label, including contrasting agents, can be used in known medical imaging techniques.

For imaging a brain tumor, a detectable label may be any molecule or agent that can emit a signal that is detectable by imaging. For example, the detectable label may be a protein, a radioisotope, a fluorophore, a visible light emitting fluorophore, infrared light emitting fluorophore, a metal, a ferromagnetic substance, an electromagnetic emitting substance a substance with a specific MR spectroscopic signature, an X-ray absorbing or reflecting substance, or a sound altering substance.

Examples of imaging methods include MRI, MR spectroscopy, radiography, CT, ultrasound, planar gamma camera imaging, single-photon emission computed tomography (SPECT), positron emission tomography (PET), other nuclear medicine-based imaging, optical imaging using visible light, optical imaging using luciferase, optical imaging using a fluorophore, other optical imaging, imaging using near infrared light, or imaging using infrared light.

Certain embodiments of the methods of the present invention further include imaging a tissue during a surgical procedure on a subject. In some embodiments, the subject is undergoing an anticancer therapy such as, but not limited to, chemotherapy, radiation therapy, surgical therapy, immunotherapy, and gene therapy.

A variety of techniques for imaging are known to those of ordinary skill in the art. Any of these techniques can be applied in the context of the imaging methods of the present invention to measure a signal from the detectable label or contrasting agent conjugated to calcitonin receptor. For example, optical imaging is one imaging modality that has gained widespread acceptance in particular areas of medicine. Examples include optical labeling of cellular components, and angiography such as fluorescein angiography and indocyanine green angiography. Examples of optical imaging agents include, for example, fluorescein, a fluorescein derivative, indocyanine green, Oregon green, a derivative of Oregon green derivative, rhodamine green, a derivative of rhodamine green, an eosin, an erytlirosin, Texas red, a derivative of Texas red, malachite green, nanogold sulfosuccinimidyl ester, cascade blue, a coumarin derivative, a naphthalene, a pyridyloxazole derivative, cascade yellow dye, dapoxyl dye.

Gamma camera imaging is contemplated as a method of imaging that can be utilized for measuring a signal derived from the detectable label. One of ordinary skill in the art would be familiar with techniques for application of gamma camera imaging. In one embodiment, measuring a signal can involve use of gamma-camera imaging of an ¹¹¹In or ^{99m}Tc conjugate, in particular ¹¹¹In-octreotide or ^{99m}Tc-somatostatin analogue.

Computerized tomography (CT) is contemplated as an imaging modality in the context of the present invention. By taking a series of X-rays from various angles and then combining them with a computer, CT made it possible to build up a three-dimensional image of any part of the body. A computer is programmed to display two-dimensional slices from any angle and at any depth. The slices may be combined to build three-dimensional representations.

In CT, intravenous injection of a radiopaque contrast agent can assist in the identification and delineation of soft tissue masses when initial CT scans are not diagnostic. Similarly, contrast agents aid in assessing the vascularity of a soft tissue lesion. For example, the use of contrast agents may aid the delineation of the relationship of a tumor and adjacent vascular structures.

CT contrast agents include, for example, iodinated contrast media. Examples of these agents include iothalamate, iohexyl, diatrizoate, iopamidol, ethiodol, and iopanoate. Gadolinium agents have also been reported to be of use as a CT contrast agent, for example, gadopentate.

Magnetic resonance imaging (MRI) is an imaging modality that uses a high-strength magnet and radio-frequency signals to produce images. In MRI, the sample to be imaged is placed in a strong static magnetic field and excited with a pulse of radio frequency (RF) radiation to produce a net magnetization in the sample. Various magnetic field gradients and other RF pulses then act to code spatial information into the recorded signals. By collecting and analyzing these signals, it is possible to compute a three-dimensional image which, like a CT image, is normally displayed in two-dimensional slices. The slices may be combined to build three-dimensional representations.

Contrast agents used in MR or MR spectroscopy imaging differ from those used in other imaging techniques. Examples of MRI contrast agents include gadolinium chelates, manganese chelates, chromium chelates, and iron particles. Both CT and MRI provide anatomical information that aid in distinguishing tissue boundaries and vascular structure.

Imaging modalities that provide information pertaining to information at the cellular level, such as cellular viability, include positron emission tomography (PET) and single-photon emission computed tomography (SPECT). In PET, a patient ingests or is injected with a radioactive substance that emits positrons, which can be monitored as the substance moves through the body.

Closely related to PET is single-photon emission computed tomography, or SPECT. The major difference between the two is that instead of a positron-emitting substance, SPECT uses a radioactive tracer that emits high-energy photons. SPECT is valuable for diagnosing multiple illnesses including coronary artery disease, and already some 2.5 million SPECT heart studies are done in the United States each year.

PET radiopharmaceuticals for imaging are commonly labeled with positron-emitters such as ¹¹C, ¹³N ¹⁵O, ¹⁸F, ⁸²Rb, ⁶²Cu, and ⁶⁸Ga. SPECT radiopharmaceuticals are commonly labeled with positron emitters such as 99mTc, ²⁰¹Tl, and ⁶⁷Ga, ¹¹¹In. Regarding brain imaging, PET and SPECT radiopharmaceuticals are classified according to blood-brain-barrier permeability, cerebral perfusion and metabolism, receptor-binding, and antigen-antibody binding (Saha et al, 1994). The blood-brain-barrier (BBB) SPECT agents, such as ^{99m}TcO4-DTPA, ²⁰¹Tl, and [⁶⁷Ga]citrate are excluded by normal brain cells, but enter into tumor cells because of altered BBB. SPECT perfusion agents such as [¹²³I]IMP, [⁹⁹ mTc]HMP AO, [⁹⁹ mTc]ECD are lipophilic agents, and therefore diffuse into the normal brain. Important receptor-binding SPECT radiopharmaceuticals include [¹²³I]QNE, [¹²³I]IBZM, and [¹²³I]iomazenil. These tracers bind to specific receptors, and are of importance in the evaluation of receptor-related diseases.

### Diagnosis, Prognosis and Prediction of Therapeutic Outcome of Brain Tumors

In one embodiment, the present invention provides a method of diagnosis, prognosis and/or prediction of a brain tumor in a subject, the method comprising detecting calcitonin receptor in brain cells of the subject, wherein the presence of calcitonin receptor is diagnostic, prognostic and/or predictive for the brain tumor.

The method of the present invention is particularly suited to medical imaging techniques. Prior to the present invention, a patient presenting with symptoms suggestive of a brain tumor would undergo an initial scan, for example, an MRI scan to determine whether there is abnormal or diseased tissue present in the brain. Should an abnormal tissue mass be detected which is suspected of being a brain tumor, a brain biopsy is taken in order to confirm whether the abnormal tissue comprises tumor cells. Using the method of the invention, a compound that binds calcitonin receptor is administered to a patient and the patient's brain imaged in order to detect calcitonin receptor expressing cells. Thus, the non-invasive method of the invention advantageously avoids the need for a brain tissue biopsy to be taken from the patient.

The diagnostic, prognostic and predictive methods of the present invention may involve a degree of quantification to determine levels of CTR present in patient samples. Such quantification is readily provided by the inclusion of appropriate control samples.

Internal controls may be included in the methods of the present invention where appropriate. In the case of medical imaging, an abnormal mass of tissue will typically be present in a brain scan, for example in an MRI scan. Thus, following administration of a compound that binds calcitonin receptor, the MRI scan will show areas of background staining or contrast (i.e., areas of normal brain tissue) that may be compared with the staining or contrast in the area containing the abnormal mass of tissue. An altered level of staining or contrast in the area containing the abnormal mass of brain tissue when compared to the normal brain tissue in the scan is indicative of the abnormal mass containing calcitonin receptor expressing tumor cells.

When the method of the invention comprises detecting calcitonin receptor in a sample obtained from a subject, a preferred internal control is one or more samples taken from one or more healthy individuals.

In the present context, the term “healthy individual” shall be taken to mean an individual who is known not to suffer from a brain tumor, such knowledge being derived from clinical data on the individual, including, but not limited to, a different diagnostic assay to that described herein.

As will be known to those skilled in the art, when internal controls are not included in each assay conducted, the control may be derived from an established data set.

Data pertaining to the control subjects are preferably selected from the group consisting of:

1. a data set comprising measurements of the presence or level of expression of CTR for a typical population of subjects known to have a brain tumor;

2. a data set comprising measurements of the presence or level of expression of CTR for the subject being tested wherein said measurements have been made previously, such as, for example, when the subject was known to be healthy or, in the case of a subject having a brain tumor, when the subject was diagnosed or at an earlier stage in disease progression;

3. a data set comprising measurements of the presence or level of expression of CTR for a healthy individual or a population of healthy individuals; and

4. a data set comprising measurements of the presence or level of expression of CTR for a normal individual or a population of normal individuals.

In the present context, the term “typical population” with respect to subjects known to have a brain tumor shall be taken to refer to a population or sample of subjects diagnosed with a brain tumor that is representative of the spectrum of the brain tumor patients. This is not to be taken as requiring a strict normal distribution of morphological or clinicopathological parameters in the population, since some variation in such a distribution is permissible. Preferably, a “typical population” will exhibit a spectrum of the brain tumor at different stages of disease progression. It is particularly preferred that a “typical population” exhibits the expression characteristics of a cohort of subjects as described herein.

As will be known to those skilled in the art, data obtained from a sufficiently large sample of the population will normalize, allowing the generation of a data set for determining the average level of calcitonin receptor expression in brain cells.

Those skilled in the art are readily capable of determining the baseline for comparison in any diagnostic assay of the present invention without undue experimentation, based upon the teaching provided herein.

### Therapeutic Methods

The present inventors have determined that CTR expression by brain tumor cells forms a component of the inflammatory process in brain tumors such as, for example, glioblastoma multiforme. The inflammatory process in brain tumors is known to play an important role in tumor expansion. In addition, the present inventors have found CTR expressing cells in brain tumors that also express CD133, a known cancer stem cell marker (Singh et al., 2003; Singh et al. (2004). Accordingly, the present inventors have for the first time identified calcitonin receptor as a marker of malignant brain tumor cells and a therapeutic target. Whereas antibodies have previously been developed that target brain tumors in a non-specific manner, for example anti-EGFR antibodies that target areas of vascularisation in a tumor, the present invention for the first time allows for the specific targeting of brain tumor cells expressing CTR.

Although the blood-brain barrier normally provides a physiologic obstruction to the delivery of therapeutic molecules to the brain, in brain tumours the blood-brain barrier is often compromised allowing access to therapeutic treatment. Thus, in one aspect, the present invention utilizes the compounds that bind calcitonin receptor without modification, relying on the binding of the compounds to CTR expressing brain cells in situ to stimulate an immune attack thereon. For example, a chimeric antibody, wherein the antigen-binding site is joined to human Fc region may be used to promote antibody-dependent mediated cytotoxicity or complement-mediated cytotoxicity.

In another aspect of the invention, the therapeutic method may be carried out using compounds that bind CTR to which a cytotoxic agent or biological response modifier is bound. Binding of the resulting conjugate to the CTR expressing cells inhibits the growth of or kills the cells, or modulates the activity, division of, or lifespan of the cells.

It will be appreciated that methods of treating brain tumors such as glioblastoma multiforme involving the use of compounds that bind calcitonin receptor may be performed in isolation or as an adjunct to known chemotherapy or radiotherapy regimes. For example, treatment with a compound that binds calcitonin receptor may be conducted in conjunction with or after treatment with drugs such as temozolomide, BCNU (Carmustine), PCV (combination of procarbazine, CCNV (Lomustine), and vincristine), carboplatin, etoposide, irinotecan, Cis-Retonoic acid, thalidomide, tamoxifen and COX-2 inhibitors.

The expression of CTR has also been associated with retardation of the cell cycle in the presence of calcitonin (Evdokiou et al., 1999; Evdokiou et al., 2000). As a consequence of cell cycle retardation, tumors expressing calcitonin receptor have reduced sensitivity to chemotherapeutics. Thus, reducing the production and/or activity of CTR in tumor cells and/or reducing the binding of calcitonin to calcitonin receptor in tumor cells will increase cell cycle activity and sensitize the cells to conventional therapies such as chemotherapy.

As used herein, the term “sensitize” refers to increasing the susceptibility of a tumor cell to a chemotherapeutic agent as a result of an increase in cell cycle activity. As would be understood in the art, an increase in cell cycle activity refers to an increase in the rate that a cell progresses through the different stages of the cell cycle (i.e. through G₁, S, G₂ and M phases) and undergoes mitosis or division.

The tumor that is sensitized to a chemotherapeutic using the method of the invention is preferably a solid tumor. Examples of solid tumors include adrenocarcinomas, glioblastomas (and other brain tumors), breast, cervical, colorectal, endometrial, prostrate, gastric, liver, lung (small cell and non-small cell), lymphomas (including non-Hodgkin's, Burkitt's, diffuse large cell, follicular and diffuse Hodgkin's), melanoma (metastatic), neuroblastoma, osteogenic sarcoma, ovarian, retinoblastoma, soft tissue sarcomas, testicular and other tumors which respond to chemotherapy.

In one embodiment, the production or activity of calcitonin receptor in a cell is reduced with a polynucleotide such as, for example, an antisense polynucleotide, catalytic polynucleotide, microRNA, or double-stranded RNA molecule such as a siRNA or shRNA.

### Cytotoxic Agents

A “cytotoxic agent” is any agent that is capable of modulating the activity, or division of, or which kills calcitonin receptor expressing cells. Suitable cytotoxic agents for use in the present invention include, but are not limited to, agents such as bacterial or plant toxins, drugs, e.g., cyclophosphamide (CTX; cytoxan), chlorambucil (CHL; leukeran), cisplatin (CisP; CDDP; platinol), busulfan (myleran), melphalan, carmustine (BCNU), streptozotocin, triethylenemelamine (TEM), mitomycin C, and other alkylating agents; methotrexate (MTX), etoposide (VP-16; vepesid), 6-mercaptopurine (6 MP), 6-thioguanine (6TG), cytarabine (Ara-C), 5-fluorouracil (5FU), dacarbazine (DTIC), 2-chlorodeoxyadenosine (2-CdA), and other antimetabolites; antibiotics including actinomycin D, doxorubicin (DXR; adriamycin), daunorubicin (daunomycin), bleomycin, mithramycin as well as other antibiotics; alkaloids such as vincristin (VCR), vinblastine, and the like; as well as other anti-cancer agents including the cytostatic agents glucocorticoids such as dexamethasone (DEX; decadron) and corticosteroids such as prednisone, nucleotide enzyme inhibitors such as hydroxyurea, and the like.

Those skilled in the art will realize that there are numerous other radioisotopes and chemocytotoxic agents that can be coupled to compounds that bind CTR by well known techniques, and delivered to destroy CTR expressing cells and/or cells in close proximity thereto. In one embodiment, the agents specifically destroy brain tumor cells (see, e.g., U.S. Pat. No. 4,542,225). Examples of photo-activated toxins include dihydropyridine- and omega-conotoxin (Schmidt et al., 1991). Examples of cytotoxic reagents that can be used include ¹²⁵I, ¹³¹I, ¹¹¹In, ¹²³I, ⁹⁹mTc, and ³²P. The antibody can be labeled with such reagents using techniques known in the art. For example, see Wenzel and Meares, Radioimmunoimaging and Radioimmunotherapy, Elsevier, N.Y. (1983) for techniques relating to the radiolabeling of antibodies (see also, Colcher et al., 1986; “Order, Analysis, Results and Future Prospective of the Therapeutic Use of Radiolabeled Antibody in Cancer Therapy”, in Monoclonal Antibodies for Cancer Detection and Therapy, Baldwin et al. (eds), pp. 303-16 (Academic Press 1985)).

In one example, the linker-chelator tiuexutan is conjugated to a compound that binds CTR, by a stable thiourea covalent bond to provide a high-affinity chelation site for Indium-111 or Yttrium-90.

The skilled person will appreciate that there are a number of bacterial or plant polypeptide toxins that are suitable for use as cytotoxic agents in the methods of the invention. These polypeptides include, but are not limited to, polypeptides such as native or modified Pseudomonas exotoxin (PE), diphtheria toxin (DT), ricin, abrin, gelonin, momordin II, bacterial RIPs such as shiga and shiga-like toxin a-chains, luffin, atrichosanthin, momordin I, Mirabilis anti-viral protein, pokeweed antiviral protein, byodin 2 (U.S. Pat. No. 5,597,569), gaporin, as well as genetically engineered variants thereof. Native PE and DT are highly toxic compounds that typically bring about death through liver toxicity. Preferably, PE and DT are modified into a form that removes the native targeting component of the toxin, e.g., domain Ia of PE and the B chain of DT. One of skill in the art will appreciate that the invention is not limited to a particular cytotoxic agent.

In some embodiments, the cytotoxic agent may be a polypeptide fused to a compound that binds CTR. Fusion polypeptides comprising a compound that binds CTR may be prepared by methods known to one of skill in the art. For example, a gene encoding an Fv region is fused to a gene encoding a cytotoxic agent. Optionally, the Fv gene is linked to a segment encoding a peptide connector. The peptide connector may be present simply to provide space between the compound that binds CTR and the cytotoxic agent or to facilitate mobility between these regions to enable them to each attain their optimum conformation. The DNA sequence comprising the connector may also provide sequences (such as primer sites or restriction sites) to facilitate cloning or may preserve the reading frame between the sequence encoding the binding moiety and the sequence encoding the cytotoxic agent. The design of such connector peptides is well known to those of skill in the art.

Generally producing fusion polypeptides involves, e.g., separately preparing the Fv light and heavy chains and DNA encoding any other protein to which they are fused and recombining the DNA sequences in a plasmid or other vector to form a construct encoding the particular desired fusion polypeptide. However, a simpler approach involves inserting the DNA encoding the particular Fv region into a construct already encoding the desired second polypeptide. The DNA sequence encoding the Fv region is inserted into the construct using techniques well known to those of skill in the art.

Compounds that bind CTR, e.g., recombinant single chain antibodies, may be fused to, or otherwise bound to the cytotoxic agent by any method known and available to those in the art. The two components may be chemically bonded together by any of a variety of well-known chemical procedures. For example, the linkage may be by way of heterobifunctional cross-linkers, e.g., SPDP, carbodiimide, glutaraldehyde, or the like. Production of various immunotoxins, as well as chemical conjugation methods, are well-known within the art (see, for example, “Monoclonal Antibody-Toxin Conjugates: Aiming the Magic Bullet,” Thorpe et al., Monoclonal Antibodies in Clinical Medicine, Academic Press, pp. 168-190 (1982); Waldmann, 1991; Vitetta et al., 1987; Pastan et al., 1986; and Thorpe et al., 1987).

It will be appreciated that methods of treating or preventing brain tumors involving the use of compounds that bind CTR may be performed in isolation or as an adjunct to known therapy regimes. For example, treatment may be conducted in conjunction with or after treatments such as chemotherapy, radiation therapy, stem cell transplant and/or immunotherapy, for example, monoclonal antibody therapy. Examples of chemotherapeutic agents used in the treatment of brain tumors include temozolomide, BCNU (Carmustine), PCV (combination of procarbazine, CCNV (Lomustine), and vincristine), carboplatin, etoposide, irinotecan, Cis-Retonoic acid, thalidomide, tamoxifen and COX-2 inhibitors. Other known chemotherapeutic agents include chlorambucil, cyclophosphamide, melphalan, daunorubicin, doxorubicin, idarubicin, mitoxantrone, methotrexate, fludarabine, cytarabine, etoposide, topotecan, prednisone, dexamethasone, vincristine and vinblastine.

### Biological Response Modifiers

A “biological response modifier” refers to any compound, particularly a polypeptide or peptide, that is able to modify, either directly or indirectly, a biological response to a calcitonin receptor expressing cell. By modifying a biological response, the activity, or division of, calcitonin receptor expressing cells is modified, or calcitonin receptor expressing cells are killed.

“Biological response modifiers” include, but are not limited to, lymphokines and cytokines (e.g., interferon gamma (IFNγ), interleukin-1 (IL-1), interleukin-2 (IL-2), interleukin-5 (IL-5), interleukin-6 (IL-6), interleukin-7 (IL-7), interleukin-10 (IL-10), interleukin-12 (IL-12), interleukin-15 (IL-15), interleukin-23 (IL-23), granulocyte macrophage colony stimulating factor (GM-CSF), and granulocyte colony stimulating factor (G-CSF)), or a growth factor (e.g., growth hormone (GH)).

Biological response modifiers may have a variety of effects on CTR expressing cells. Among these effects are increased cell killing by direct action as well as increased cell killing by increased host defence mediated processes. For example, conjugation of a compound that binds CTR to these biological response modifiers will allow selective localization within brain tumor cells, and hence improved anti-proliferative effects while suppressing non-specific effects leading to toxicity of non-target cells.

### Antisense Polynucleotides

The term “antisense polynucleotide” shall be taken to mean a DNA or RNA, or combination thereof, molecule that is complementary to at least a portion of a mRNA molecule encoding calcitonin receptor and capable of interfering with a post-transcriptional event such as mRNA translation. The use of antisense methods is well known in the art (see for example, G. Hartmann and S. Endres, Manual of Antisense Methodology, Kluwer (1999)).

An antisense polynucleotide useful for the invention will hybridize to a target polynucleotide under physiological conditions. As used herein, the term “an antisense polynucleotide which hybridises under physiological conditions” means that the polynucleotide (which is fully or partially single stranded) is at least capable of forming a double-stranded polynucleotide with mRNA encoding a protein, in a cell.

Antisense molecules may include sequences that correspond to the structural genes or for sequences that effect control over the gene expression or splicing event. For example, the antisense sequence may correspond to the targeted coding region of the calcitonin receptor gene, or the 5′-untranslated region (UTR) or the 3′-UTR or combination of these. It may be complementary in part to intron sequences, which may be spliced out during or after transcription, preferably only to exon sequences of the target gene. In view of the generally greater divergence of the UTRs, targeting these regions provides greater specificity of gene inhibition.

The length of the antisense sequence should be at least 19 contiguous nucleotides, preferably at least 50 nucleotides, and more preferably at least 100, 200, 500 or 1000 nucleotides. The full-length sequence complementary to the entire gene transcript may be used. The length is most preferably 100-2000 nucleotides. The degree of identity of the antisense sequence to the targeted transcript should be at least 90% and more preferably 95-100%. The antisense RNA molecule may of course comprise unrelated sequences which may function to stabilize the molecule.

### Catalytic Polynucleotides

The term catalytic polynucleotide/nucleic acid refers to a DNA molecule or DNA-containing molecule (also known in the art as a “deoxyribozyme”) or an RNA or RNA-containing molecule (also known as a “ribozyme”) which specifically recognizes a distinct substrate and catalyzes the chemical modification of this substrate. The nucleic acid bases in the catalytic nucleic acid can be bases A, C, G, T (and U for RNA).

Typically, the catalytic nucleic acid contains an antisense sequence for specific recognition of a target nucleic acid, and a nucleic acid cleaving enzymatic activity (also referred to herein as the “catalytic domain”). The types of ribozymes that are particularly useful in this invention are the hammerhead ribozyme (Perriman et al., 1992) and the hairpin ribozyme (Shippy et al., 1999).

The ribozymes useful for this invention and DNA encoding the ribozymes can be chemically synthesized using methods well known in the art. The ribozymes can also be prepared from a DNA molecule (that upon transcription, yields an RNA molecule) operably linked to an RNA polymerase promoter, e.g., the promoter for T7 RNA polymerase or SP6 RNA polymerase. When the vector also contains an RNA polymerase promoter operably linked to the DNA molecule, the ribozyme can be produced in vitro upon incubation with RNA polymerase and nucleotides. In a separate embodiment, the DNA can be inserted into an expression cassette or transcription cassette. After synthesis, the RNA molecule can be modified by ligation to a DNA molecule having the ability to stabilize the ribozyme and make it resistant to RNase.

As with antisense polynucleotides described herein, catalytic polynucleotides useful for the invention should also be capable of hybridizing a target nucleic acid molecule under “physiological conditions”, namely those conditions within a cell (especially conditions in an animal cell such as a human cell).

### RNA Interference

The terms “RNA interference”, “RNAi” or “gene silencing” refer generally to a process in which a double-stranded RNA molecule reduces the expression of a nucleic acid sequence with which the double-stranded RNA molecule shares substantial or total homology. However, it has more recently been shown that RNA interference can be achieved using non-RNA double stranded molecules (see, for example, US 20070004667).

The methods of the present invention utilise nucleic acid molecules comprising and/or encoding double-stranded regions for RNA interference. The nucleic acid molecules are typically RNA but may comprise chemically-modified nucleotides and non-nucleotides.

The double-stranded regions should be at least 19 contiguous nucleotides, for example about 19 to 23 nucleotides, or may be longer, for example 30 or 50 nucleotides, or 100 nucleotides or more. The full-length sequence corresponding to the entire gene transcript may be used. Preferably, they are about 19 to about 23 nucleotides in length.

The degree of identity of a double-stranded region of a nucleic acid molecule to the targeted transcript should be at least 90% and more preferably 95-100%. The nucleic acid molecule may of course comprise unrelated sequences which may function to stabilize the molecule.

The term “short interfering RNA” or “siRNA” as used herein refers to a nucleic acid molecule which comprises ribonucleotides capable of inhibiting or down regulating gene expression, for example by mediating RNAi in a sequence-specific manner, wherein the double stranded portion is less than 50 nucleotides in length, preferably about 19 to about 23 nucleotides in length. For example the siRNA can be a nucleic acid molecule comprising self-complementary sense and antisense regions, wherein the antisense region comprises nucleotide sequence that is complementary to nucleotide sequence in a target nucleic acid molecule or a portion thereof and the sense region having nucleotide sequence corresponding to the target nucleic acid sequence or a portion thereof. The siRNA can be assembled from two separate oligonucleotides, where one strand is the sense strand and the other is the antisense strand, wherein the antisense and sense strands are self-complementary.

As used herein, the term siRNA is meant to be equivalent to other terms used to describe nucleic acid molecules that are capable of mediating sequence specific RNAi, for example micro-RNA (miRNA), short hairpin RNA (shRNA), short interfering oligonucleotide, short interfering nucleic acid (siNA), short interfering modified oligonucleotide, chemically-modified siRNA, post-transcriptional gene silencing RNA (ptgsRNA), and others. In addition, as used herein, the term RNAi is meant to be equivalent to other terms used to describe sequence specific RNA interference, such as post transcriptional gene silencing, translational inhibition, or epigenetics. For example, siRNA molecules as described herein can be used to epigenetically silence genes at both the post-transcriptional level or the pre-transcriptional level. In a non-limiting example, epigenetic regulation of gene expression by siRNA molecules as described herein can result from siRNA mediated modification of chromatin structure to alter gene expression.

By “shRNA” or “short-hairpin RNA” is meant an RNA molecule where less than about 50 nucleotides, preferably about 19 to about 23 nucleotides, is base paired with a complementary sequence located on the same RNA molecule, and where said sequence and complementary sequence are separated by an unpaired region of at least about 4 to about 15 nucleotides which forms a single-stranded loop above the stem structure created by the two regions of base complementarity.

Included shRNAs are dual or bi-finger and multi-finger hairpin dsRNAs, in which the RNA molecule comprises two or more of such stem-loop structures separated by single-stranded spacer regions.

Once designed, the nucleic acid molecules comprising a double-stranded region can be generated by any method known in the art, for example, by in vitro transcription, recombinantly, or by synthetic means.

Modifications or analogs of nucleotides can be introduced to improve the properties of the nucleic acid molecules. Improved properties include increased nuclease resistance and/or increased ability to permeate cell membranes. Accordingly, the terms “nucleic acid molecule” and “double-stranded RNA molecule” includes synthetically modified bases such as, but not limited to, inosine, xanthine, hypoxanthine, 2-aminoadenine, 6-methyl-, 2-propyl- and other alkyl-adenines, 5-halo uracil, 5-halo cytosine, 6-aza cytosine and 6-aza thymine, pseudo uracil, 4-thiuracil, 8-halo adenine, 8-aminoadenine, 8-thiol adenine, 8-thiolalkyl adenines, 8-hydroxyl adenine and other 8-substituted adenines, 8-halo guanines, 8-amino guanine, 8-thiol guanine, 8-thioalkyl guanines, 8-hydroxyl guanine and other substituted guanines, other aza and deaza adenines, other aza and deaza guanines, 5-trifluoromethyl uracil and 5-trifluoro cytosine.

MicroRNA regulation is a specialized branch of the RNA silencing pathway that evolved towards gene regulation, diverging from conventional RNAi/PTGS. MicroRNAs are a specific class of small RNAs that are encoded in gene-like elements organized in a characteristic inverted repeat. When transcribed, microRNA genes give rise to stem-looped precursor RNAs from which the microRNAs are subsequently processed. MicroRNAs are typically about 21 nucleotides in length. The released miRNAs are incorporated into RISC-like complexes containing a particular subset of Argonaute proteins that exert sequence-specific gene repression (see, for example, Millar and Waterhouse, 2005; Pasquinelli et al., 2005; Almeida and Allshire, 2005).

### Calcitonin Receptor Agonists

The present inventors have demonstrated that administering a calcitonin receptor agonist to cells expressing calcitonin receptor alters the cell cycle and reduces cell proliferation. By activating intracellular signals via calcitonin receptor, the tumor cell cycle is slowed thereby reducing the proliferation of tumor cells. By “reducing the proliferation of tumor cells” it is meant that the rate of tumor cell proliferation is decreased when compared to tumor cells from a tumor of a similar stage or grade which have not been treated with a calcitonin receptor agonist. The degree of decrease in the proliferation of tumor cells will vary with the nature and quantity of the agonist present, but will be evident e.g., as a detectable decrease in the proliferation of tumor cells; desirably a degree of decrease greater than 5%, 10%, 33%, 50%, 75%, 90%, 95% or 99% as compared to the proliferation of tumor cells in the absence of the compound.

By reducing the proliferation of tumor cells, the method of treatment advantageously reduces tumor expansion. As used herein, the phrase “reduces tumor expansion” means that the expansion, growth or progression of a tumor is decreased when compared to a tumor of a similar stage or grade which has not been treated with a CTR agonist. The degree of decrease in the expansion, growth or progression of the tumor will vary with the nature and quantity of the agnonist present, but will be evident e.g., as a detectable decrease in the expansion, growth or progression of a tumor; desirably a degree of decrease greater than 5%, 10%, 33%, 50%, 75%, 90%, 95% or 99% as compared to the expansion, growth or progression of a tumor in the absence of the compound.

Accordingly, a brain tumor may be treated by administering a calcitonin receptor agonist. As used herein, the term “calcitonin receptor agonist” refers to a compound that is capable of binding and signalling via calcitonin receptor. As will be known to the person skilled in the art, suitable CTR agonists include calcitonin (CT) and calcitonin binding analogues. Examples of calcitonin binding analogues are known in the art and include those described in, for example, Boros et al. (2005) and Dong et al. (2009).

The methods of treatment of the invention comprising administering a calcitonin receptor agonist may be used in conjunction with conventional therapies such as radiotherapy and chemotherapy suitable for treating brain tumors as known in the art.

### Pharmaceutical Compositions, Dosages, and Routes of Administration

Compositions comprising a compound that binds CTR together with an acceptable carrier or diluent are useful in the methods of the present invention.

Therapeutic compositions can be prepared by mixing the desired compounds having the appropriate degree of purity with optional pharmaceutically acceptable carriers, excipients, or stabilizers (Remington's Pharmaceutical Sciences, 16th edition, Osol, A. ed. (1980)), in the form of lyophilized formulations, aqueous solutions or aqueous suspensions. Acceptable carriers, excipients, or stabilizers are preferably nontoxic to recipients at the dosages and concentrations employed, and include buffers such as Tris, HEPES, PIPES, phosphate, citrate, and other organic acids; antioxidants including ascorbic acid and methionine; preservatives (such as octadecyldimethylbenzyl ammonium chloride; hexamethonium chloride; benzalkonium chloride, benzethonium chloride; phenol, butyl or benzyl alcohol; alkyl parabens such as methyl or propyl paraben; catechol; resorcinol; cyclohexanol; 3-pentanol; and m-cresol); low molecular weight (less than about 10 residues) polypeptides; proteins, such as serum albumin, gelatin, or immunoglobulins; hydrophilic polymers such as polyvinylpyrrolidone; amino acids such as glycine, glutamine, asparagine, histidine, arginine, or lysine; monosaccharides, disaccharides, and other carbohydrates including glucose, mannose, or dextrins; sugars such as sucrose, mannitol, trehalose or sorbitol; salt-forming counter-ions such as sodium; and/or non-ionic surfactants such as TWEEN™, PLURONICS™ or polyethylene glycol (PEG).

Additional examples of such carriers include ion exchangers, alumina, aluminum stearate, lecithin, serum proteins, such as human serum albumin, buffer substances such as glycine, sorbic acid, potassium sorbate, partial glyceride mixtures of saturated vegetable fatty acids, water, salts, or electrolytes such as protamine sulfate, disodium hydrogen phosphate, potassium hydrogen phosphate, sodium chloride, colloidal silica, magnesium trisilicate, polyvinyl pyrrolidone, and cellulose-based substances.

Therapeutic compositions to be used for in vivo administration should be sterile. This is readily accomplished by filtration through sterile filtration membranes, prior to or following lyophilization and reconstitution. The composition may be stored in lyophilized form or in solution if administered systemically. If in lyophilized form, it is typically formulated in combination with other ingredients for reconstitution with an appropriate diluent at the time for use. An example of a liquid formulation is a sterile, clear, colorless unpreserved solution filled in a single-dose vial for subcutaneous injection.

Therapeutic compositions generally are placed into a container having a sterile access port, for example, an intravenous solution bag or vial having a stopper pierceable by a hypodermic injection needle. The compositions are preferably administered parenterally, for example, as intravenous injections or infusions or administered into a body cavity.

The growth of CTR expressing cells may be inhibited or reduced by administering to a subject in need of the treatment an effective amount of a composition comprising a compound that binds CTR. The compound may be administered in an amount of about 0.001 to 2000 mg/kg body weight per dose, and more preferably about 0.01 to 500 mg/kg body weight per dose. Repeated doses may be administered as prescribed by the treating physician.

Single or multiple administrations of the compositions are administered depending on the dosage and frequency as required and tolerated by the patient. The dosage and frequency will typically vary according to factors specific for each patient depending on the specific therapeutic or prophylactic agents administered, the severity and type of brain tumor, the route of administration, as well as age, body weight, response, and the past medical history of the patient. Suitable regimens can be selected by one skilled in the art by considering such factors and by following, for example, dosages reported in the literature and recommended in the Physician's Desk Reference (56^{th} ed., 2002). Generally, the dose is sufficient to treat or ameliorate symptoms or signs of disease without producing unacceptable toxicity to the patient.

In any treatment regimen, the therapeutic composition may be administered to a patient either singly or in a cocktail containing other therapeutic agents, compositions, or the like, including, but not limited to, immunosuppressive agents, tolerance-inducing agents, potentiators and side-effect relieving agents. Examples of immunosuppressive agents include prednisone, melphalain, prednisolone, DECADRON (Merck, Sharp & Dohme, West Point, Pa.), cyclophosphamide, cyclosporine, 6-mercaptopurine, methotrexate, azathioprine and i.v. gamma globulin or their combination. Preferred potentiators include monensin, ammonium chloride, perhexyline, verapamil, amantadine and chloroquine. All of these agents are administered in generally accepted efficacious dose ranges such as those disclosed in the Physician's Desk Reference, 41st Ed., Publisher Edward R. Barnhart, N.J. (1987).

### EXAMPLES

### Example 1

### Materials and Methods

### Human Brain Tissues and Preparation

In total the tissues with malignancies from fourteen patients diagnosed with glioblastoma multiforme (GBM) were investigated in this study. A detailed description of the pathophysiological characteristics of the GBM tumours of these patients has been recorded and these are rated grade IV according to the WHO guidelines.

Studies utilising archival remnants of GBM from resected brain tumours received ethics approval from the Alfred Hospital Ethics Committee (Project #53/07).

The GBMs were resected from the patients using standard micro-neurosurgical techniques, and a small portion of each tumour was sent to anatomical pathology for routine histological analysis and stored after processing as archival material.

Prior to immunohistochemical staining the segments of brain tissue were either fixed in buffered formalin (16 hours, room-temperature for archival material, FIGS. 2 and 4) or frozen on dry ice prior to fixation for 4 hours in fresh 4% para-formaldehyde/phosphate buffered saline (PBS, FIG. 3), and then processed and embedded in paraffin blocks. Sections were cut 7 μm thick and mounted on Superfrost Plus (Menzel-Glaser, Germany) glass slides. For confocal studies upon resection in surgery samples were immediately fixed in 4% PFA/PBS overnight prior to infusion with OCT. Frozen sections 16 μm thick were cut on a cryostat at −20° C.

### Cell Lines

Stable transfectants of the monkey kidney cell line COS-7 were cultured in high glucose Dulbecco's modified Eagle's medium (D-MEM, Invitrogen, US) plus 10% fetal bovine serum (FBS, Invitrogen, US) and incubated in a humidified 37° C. incubator with 5% CO₂. Polyclonal COS-7 cell lines, stably expressing cMyc tagged hCTR or a vector control were derived from the parental line by a combination of selection, using 10 μg/mL puromycin (Invivogen, USA), and FACS of CTR positive cells (negative cells in the case of vector control) and then maintained in high glucose D-MEM plus 10% FBS with 10 μg/mL puromycin (Invivogen, US) in a humidified 37° C. incubator with 5% CO₂. Transgene expression in all cases was confirmed by flow cytometry (not shown) using the anti-cMyc antibody 9E10.

A 3T3 cell line, used to make enriched plasma membranes, stably expressing cMyc tagged hCTR, was derived from the parental line using the flpIN system (Invitrogen, USA) and selected using 200 μg/mL hygromycin (Roche, USA). It was maintained in high glucose D-MEM plus 10% FBS with 200 μg/mL hygromycin in a humidified 37° C. incubator with 5% CO₂.

The glioblastoma cell line A172 was maintained in DMEM/10% FBS.

### Antibodies

These studies involved the use of two mouse monoclonal anti-human CTR antibodies, one directed against an extra-cellular epitope (MAb 9B4, Welcome Receptor Antibodies Melbourne, Australia (MRA) refer to WO 2009/039584; distributed as MCA5749 by AbD Serotec, UK), and the second against a cytoplasmic epitope (MAb 31-01, WRA, also known as MCA 2191, AbD Serotec, UK).

The monoclonal cell line isolated to synthesize the MAb 9B4 antibody (IgG2A), which was raised against an extra-cellular epitope, is maintained at the ECACC storage and reference facility, Health Protection Agency, Porton Down, Wiltshire SP4 OJG, United Kingdom. The second monoclonal antibody (MAb 31-01 or MCA 2191, IgG2A) was raised against an epitope within the carboxyl terminal of human CTR (DIPIYICHQELRNEPANN (SEQ ID NO:3) using standard techniques for monoclonal production as described (Tikellis et al., 2003; Wookey et al., 2008).

Previous studies using MCA 2191 have been published (Wookey et al., 2008; Silvestris et al., 2008; Wookey et al., 2009 in: Hay D, Dickerson I, eds. The calcitonin gene-related peptide family; form, function and future perspectives, Springer 2009) and these have demonstrated the high quality of this antibody in immunohistochemical experiments which yielded images with high signal to background ratios. These antibodies have also been tested by fluorescence activated cell sorting (FACS) analysis of cells from patients diagnosed with multiple myeloma (Silvestris et al., 2008) and cell lines derived from human leukaemia such as the myelogenous K-562 known to express CTR (Silvestris et al., 2008; Mould and Pondel, 2003; also unpublished FACS data for antibodies MCA 2191 and MAb 9B4).

Finally, the inventors have tested both mouse monoclonal antibodies MCA 2191 and MAb 9B4 in ELISA assays against the antigenic peptides used to raise these antibodies. MCA 2191 (purified, 1 mg/mL) could be diluted to 1:40,000 for 50% colour formation against the human sequence listed above and for MAb 9B4 (purified, 1 mg/mL) a similar response was observed with a dilution of 1:20,000 against the antigenic peptide used for its production.

In FIG. 3 the additional antibodies used included an anti-human calcitonin receptor-like receptor (anti-CLR) antibody (affinity purified rabbit polyclonal, AB 9414, Chemicon/Millipore, USA) diluted 1:500 and a mouse monoclonal anti-GFAP antibody (MAB 3402, Chemicon/Millipore, USA) diluted 1:3000.

For the confocal analysis and multi-labeling immuno-fluorescence experiments the antibodies used included the anti-CTR antibodies MCA2191 and 9B4, anti-GFAP antibody (Z0334, DAKO, Denmark), anti-nestin antibody (MAB1259, R&D Systems, US) and CD133 antibody (ab19898, Abcam, UK).

### Immunoblots

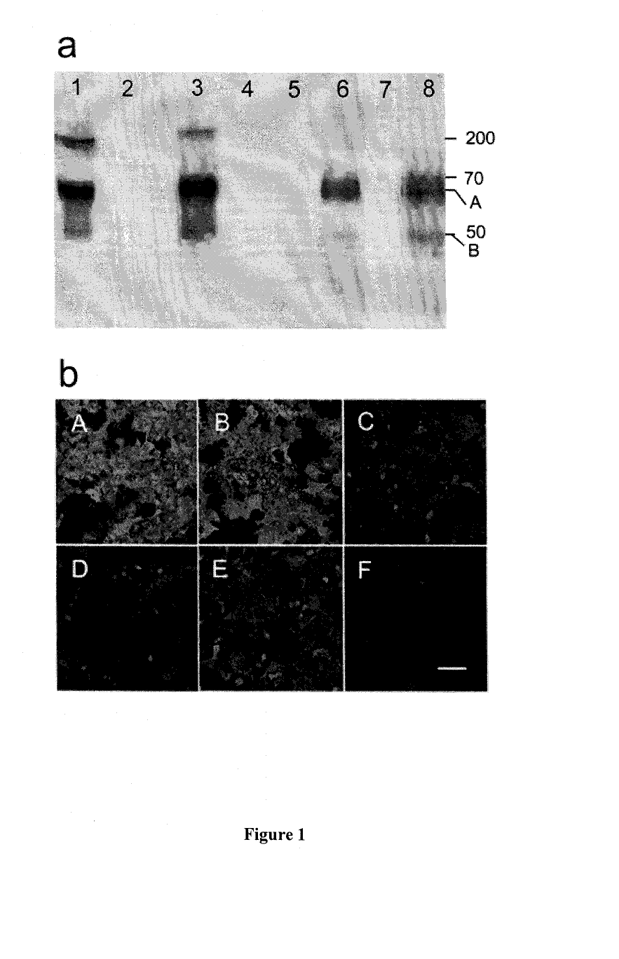

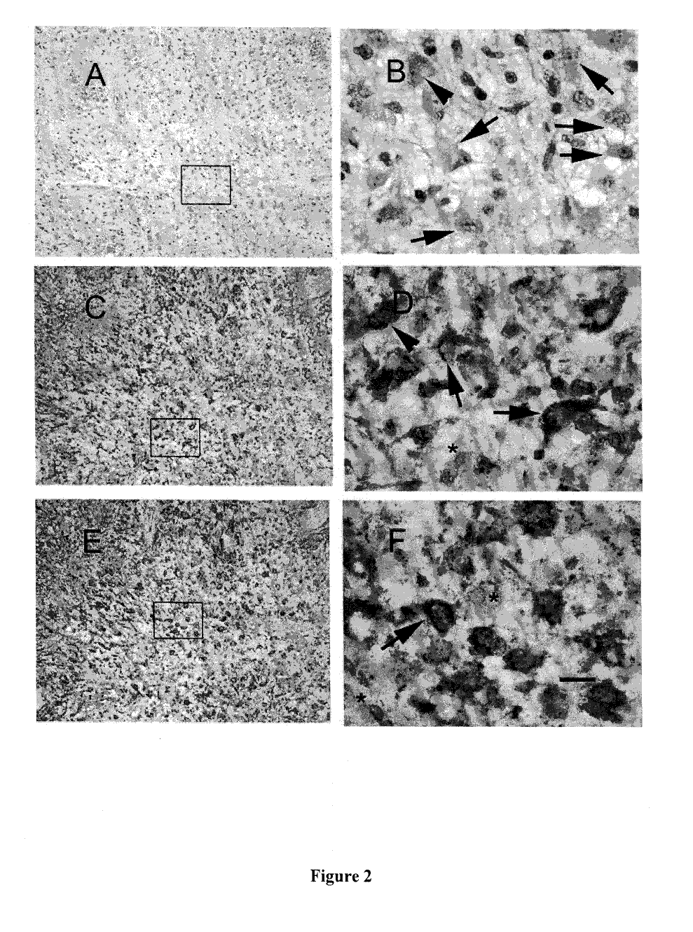

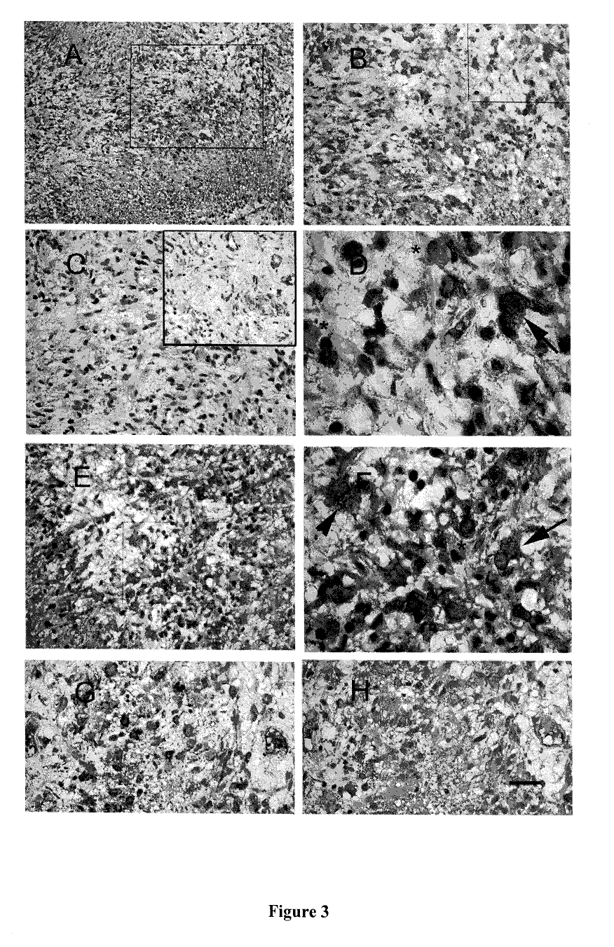

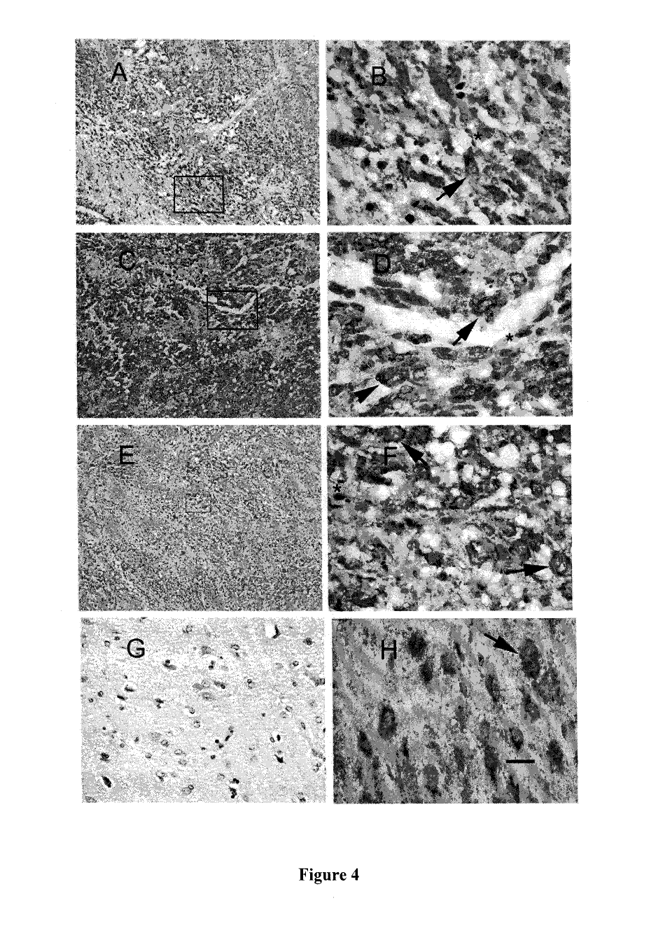

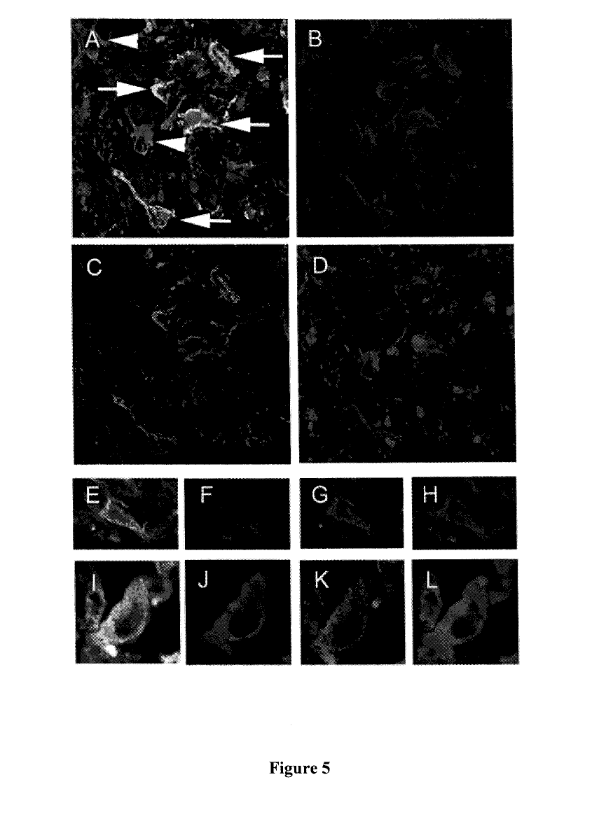

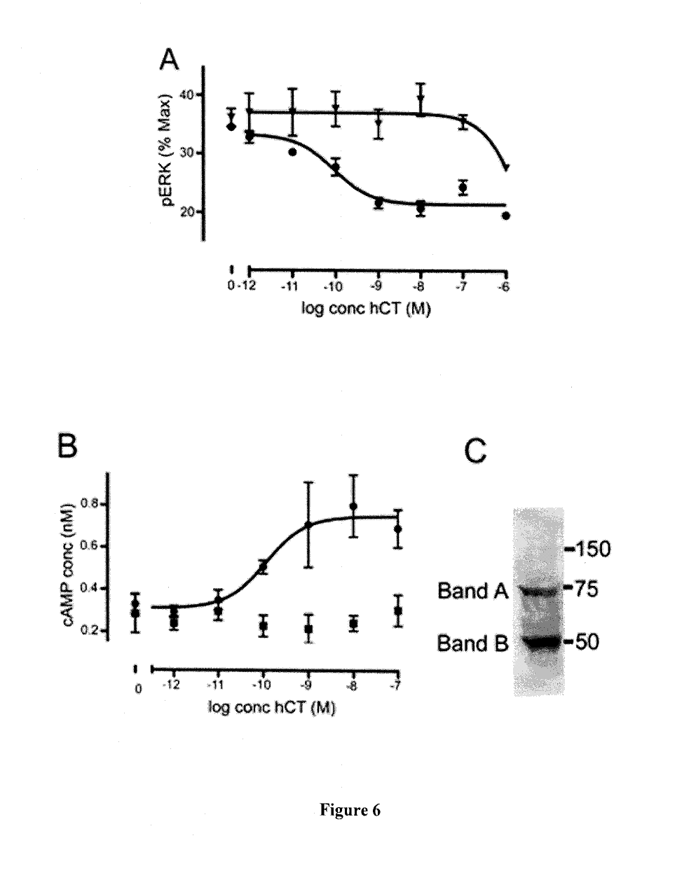

### A. Crude Membrane Preparations for the Analysis of Stable Transfectants of COS-7 (FIG. 1)

COS-7 transfectants were grown to 80% confluence. Cells were detached with 5 mL Versene per flask, 5 mL 1% BSA/PBS added and the cells centrifuged at 300 g for 10 minutes. Crude membrane preparations were made as described previously (Tikellis et al., 2003). Aliquots were stored at −80° C. Protein was determined BCA protein assay kit (ThermoScientific, Rockford, US).

Samples were solubilised in standard sample buffer, heated for 5 minutes at 50° C. and loaded onto the PAGE-SDS acrylamide gel (3% stacking/8% resolving) with Invitrogen Benchmark pre-stained protein standards. The amounts of crude membrane protein loaded onto each lane were 25 μg (lanes 1-4). The resolved proteins were transferred from the 1.5 mm gel (semi-dry blot, BioRad Transblot SD) onto 0.2 μm PVDF membrane (BioRad, US) over 1 hour at 15 volts.

The identification of protein bands was achieved using the Pierce ECL protocol (ThermoScientific, Rockford, US) and the final concentrations of primary mouse antibodies included 10 μg/mL for MCA2191 and 9B4 and secondary antibody, goat polyclonal anti-mouse/HRP (1:7000 of DAKO P0447). The bands were detected using a LAS 3000 Chemiluminescence detector (FujiFilm) and software (Image Reader LAS 3000, version 2.0, FujiFilm).

### B. Plasma Membrane Preparations for the Analysis of 3T3 Stable Transfectants (FIG. 1)

Transformed mouse embryonic fibroblast cell line flpIN-3T3 (Invitrogen, USA) was cultured in high glucose Dulbecco's modified Eagle's medium (D-MEM) plus 10% fetal bovine serum (FBS) with 100 μg/mL zeocin in a humidified 37° C. incubator with 5% CO₂. A cell line, stably expressing cMyc tagged hCTRaLeu was derived from the parental line using the flpIN system (Invitrogen, USA) and selected using 200 μg/mL hygromycin (Roche, USA). The derived cell line was then maintained in high glucose D-MEM plus 10% FBS with 200 μg/mL hygromycin in a humidified 37° C. incubator with 5% CO₂. Transgene expression was confirmed by flow cytometry (not shown) using the anti-cMyc antibody 9E10.

Enriched plasma membrane from parental and hCTRaLeu expressing flpIN-3T3 cells was prepared according to the published protocol (Hoessli et al., 1983). Media was aspirated from cells and cells rinsed with PBS. Cells were harvested in a final volume of 10 mL, ice-cold homogenisation buffer (Wittig et al., 2006) (6.6 mM imidazole/83 mM sucrose pH 7.0 containing 100 μM phenylmethane sulfonyl fluoride (Sigma, USA) and 1:1000 dilution of protease inhibitor cocktail (P8340, Sigma, USA)). Cells were disrupted with 3×10 sec bursts of a polytron homogeniser on ice (10 mm blade, power setting 3 (Pro Scientific, USA)). Cell homogenate was overlayed on a discontinuous sucrose density gradient consisting of 8 mL 60%, 8 mL 40%, 8 mL 10% sucrose in homogenisation buffer in Sorvall 36 mL polyallomer tubes (Thermo Scientific, USA). The sucrose gradient was centrifuged at 23,500 rpm (RCF at rₘₐₓ of 102,400) for 3 hrs at 4° C. using a Sorvall Surespin 630/36 rotor (Thermo Scientific, USA). Enriched plasma membrane (5 mL) was recovered from the 40%/10% interface and diluted with homogenisation buffer to 17 mL in Sorvall 17 mL polyallomer tubes and pelleted by centrifugation at 30.00 rpm (RCF at rₘₐₓ of 166,880) for 30 mins at 4° C. in a Sorvall Surespin 630/17 (Thermo Scientific, USA). The final pellet was resuspended in homogenisation buffer and protein concentration assayed by BCA (Pierce, USA).

Protein (60 μg) from a plasma membrane preparation was loaded onto an 8% SDS-PAGE mini-gel and electrophoresed at 100V. Gels were transferred to polyvinylidene difluoride membrane (BioRad, USA) overnight at 4° C. at 30V. After transfer blots were rinsed in PBS-T and blocked with 5% BSA/PBS-T for 30 mins. Blots were then incubated for 90 mins with 1° antibodies as indicated, washed 3×PBS-T, incubated for 45 mins with 2° antibody in dark, washed 3×PBS-T in dark and imaged. Precision molecular weight standards (BioRad, USA) were used to estimate sizes. Primary antibodies (MCA 2191 and MAb 9B4) were used at a concentration of 1 μg/mL in 1% BSA/PBS-T plus 0.02% azide. Secondary, goat anti-mouse AF-647 (Molecular Probes, USA) was used at 0.5 μg/mL in PBS-T plus 0.02% azide. Immunoblots were imaged on a Typhoon (GE Lifesciences, USA), using 633 nM laser and 670/30 nM emission filter.

### C. Total Cell Lysates of A172 Cells (FIG. 6)

Cells were grown to 50% confluence. Cells were detached with 5 mL Versene per flask, 5 mL 1% BSA/PBS added and the cells centrifuged at 300 g for 10 minutes. Total-cell lysates were made as described previously (Tikellis, 2003). Loading and blotting conditions were outlined above for the crude membrane preparations.

### Immunohistochemistry (IHC)

All immunohistochemical staining was performed using the CSA II amplification kit (DAKO, Denmark) as described in the manufacturer's protocol except for the incubation step with the primary antibody. In the present study, the slides were incubated overnight at 6° C. with primary antibody. Colour was developed using DAB (diaminobenzidine). The counterstain was haematoxylin (Amber Scientific, Australia) and eosin in the case of FIG. 2, panels A and B.

In the IHC protocol followed to generate images as shown in FIGS. 2 and 4 MCA 2191 was diluted 1:3000 & 1:4000 respectively, and MAb 9B4, 1:400 (FIG. 2).

In FIG. 3 MCA 2191 was used at a dilution of 1:2000 (panels A, B and D) and 1:3000 (panels G and H). In addition, antibodies used included an anti-human CLR antibody (affinity purified rabbit polyclonal, AB 9414, Chemicon/Millipore, USA) diluted 1:500 and a mouse monoclonal anti-GFAP antibody (MAB 3402, Chemicon/Millipore, USA) diluted 1:3000.

The neutralisation protocol used to generate the image in FIG. 3, panel H involved the addition of a 100-fold molar excess of the antigenic peptide (listed above) and incubated at room temperature for 1 hour. Both test (with peptide, panel H) and control (no peptide, panel G) were centrifuged for 5 minutes at 10,000 rpm before layering onto adjacent serial sections. Colour was developed as described above.

### Processing of Images and Generation of Data

Images were captured using an Olympus BX50 microscope and a Leica DFC 490 digital camera using the associated software package (Leica version 2.8.1) before processing the images in Photoshop Elements (6.0) to produce the final forms as shown in the Figures.

Fields that were investigated for each patient sample were selected for the high density of malignant cells in regions surrounding complex glomeruloid structures. The intensity of stain was not quantified, but instead, the relative numbers of positive or negative-staining malignant cells (see FIGS. 2-4) were determined by counting in a given area.

### Statistical Analysis

Analysis was performed using Chi-square on a one way classification (MedCalc, www.medcalc.be).

### Confocal Microscopy of COS-7 Cell Lines

COS-7 cell lines were grown in DMEM+10% FBS medium to 50-80% confluence on 4-well glass slides (NUN154526, Lab Tek II chamber slides) in a humidified incubator (5% CO₂) at 37° C. then washed with DMEM alone. Cells were fixed with 4% pfa/PBS at 6° C. overnight and washed twice with PBS. Cells were blocked (1:10 dilution, FcReceptor blocker [Miltenyi Biotec, Germany] in 1% Triton X-100/PBS) and incubated at RT for 1 hour.

Slides of fixed cells were then incubated overnight at 6° C. with either the primary anti-human CTR antibodies MCA2191 or 9B4 (5 μg/mL) or the mouse monoclonal IgG2A isotype control (5 μg/mL, #349050, BD Biosciences, US). The secondary antibody used was goat anti-mouse Alexa fluor 488 (4 μg/mL, Molecular Probes, Invitrogen, USA). The samples were then mounted using Invitrogen DAPI aqueous mount (Prolong Gold) and dried at 6° C. for several days in the dark.

The samples were imaged by confocal microscopy (Objectives X20) on a Zeiss Imager Z1/LSM 510 Meta confocal laser scanning system using Zen software. DAPI (405 nm) was used to visualise the nuclei. Images (LSM format) were captured in a single focal plane (optical sections of 0.7 μm nominal thickness) or using the Z-series feature where ˜15 optical sections were compressed (devolution using LSM Image Browser, Zeiss) to create a single plane image (TIFF format) equivalent to approximately 20 μm tissue thickness.

Immuno-Fluorescence with Multi-Channel Detection Using Confocal Microscopy

Fixed tissue (4% pfa/PBS overnight) was cut using a cryostat and 16 μm sections mounted on Superfrost plus (Menzel-Glaser, Germany)) glass slides. Sections were washed twice in PBS to clear OCT and once in PBS/1% Triton X-100, prior to blocking in 5% normal goat serum (NGS)/PBS/1% Triton X-100 for 1 hour. Primary antibodies were diluted 1:100 MCA2191 (mouse IgG2A), 1:100 anti-nestin antibody (mouse IgG1) and 1:100 anti-GFAP (rabbit IgGs) and mixed together in the block buffer 5% NGS/PBS/1% Triton X-100 and incubated overnight at 6° C. The sections were washed three times in PBS and secondary antibodies (each 1:500 of goat anti-mouse IgG1:Alexa 633, goat anti-mouse IgG2A:Alexa 568, goat anti-rabbit IgG:Alexa 488, Invitrogen, USA) diluted in PBS were mixed together and applied to sections for 1 hour at room temperature. The sections were washed three times in PBS before mounting with Prolong Gold with the nuclear stain DAPI (405 nm, Invitrogen, USA). The sections were dried in the dark at room temperature for several days prior to confocal microscopy.

The samples were imaged by confocal microscopy (Objectives X100 oil) on a Zeiss Imager Z1/LSM 510 Meta confocal laser scanning system using Zen software as described above and multiple Z-plane images processed by devolution.

### Example 2

### Results

Analyses with immunoblots of membrane protein are shown in FIG. 1A. Immunoblots with preparations of total membrane were used in this study to characterize the bands detected by both anti-CTR antibodies (MCA2191 and 9B4), firstly in membranes from COS-7/CTR+ and COS-7/CTR− as controls (Lanes 1-4). The major band found in COS-7/CTR+ runs with an apparent molecular weight of about 70 kD (Band A in FIG. 1A) with a minor band (Band B) at about 50 kD.

Further blots using both MCA 2191 and 9B4, but this time with preparations of plasma membrane from stable, transfected 3T3 cells (vector alone [lanes 5 and 7]) and with expression of hCTR (lanes 6 [MCA2191] and 8 [9B4]) demonstrated that both antibodies recognized a similar protein target with an apparent molecular weight of approximately 67 kDaltons. There is also a weaker band at 52 kD which is likely to represent a partially or fully de-glycosylated form of CTR (Nygaard et al., 1997). Together these data are consistent with a single target in the membrane being hCTR and demonstrate the fidelity of the antibodies in immunoblots.

In FIG. 1B are shown Z-plane stacked confocal images of the controls COS-7/CTR+(panels A-C) and COS-7/CTR− (panels D-F). These cell lines had been grown in plastic chambers on glass slides and were stained using the primary anti-CTR antibodies 9B4 (panels A, D) and MCA2191 (panels B, E). During processing the samples had been washed in 1% Triton X-100 which may render the cells permeable and expose the cytoplasmic epitope 1 of CTR (target of MCA 2191). As negative controls, a non-specific IgG2A isotype controls were also prepared for each cell line (panels C, F). Each image in the set reflects the intensity of the field as viewed under the fluorescence microscope.

Thin sections of GBM tissue (fixed buffered formalin from patient #2) are shown in FIG. 2 with images from sections counter-stained with haematoxylin and eosin at low (panel A) and high magnification (panel B). At low magnification, malignant cells are found to be concentrated around complex glomeruloid structures or vascular spaces. Examples of malignant cells are highlighted with arrows. In these representative images from sections of each GBM tumour, cells displayed extended cytoplasm and large, relatively lightly stained nuclei with condensed linear chromatin formations and often prominent nucleoli.

There is a concentration of CTR-immunoreactive (CTR-ir) cells in the region of blood vessels shown at low magnification in panels C and E.

Also in FIG. 2 are shown representative images with MCA 2191 (cytoplasmic epitope, panels C and D) and 9B4 (extra-cellular epitope, panels E and F). Both antibodies stain cell bodies and processes of cells that display similar cellular and nuclear morphologies, characteristic of malignant cells (see panel B). There are several examples of staining concentrated towards the outer profile of CTR-ir cells suggesting concentration near or within the plasma membrane.

In FIG. 3 are shown images of one malignant tumour (patient #4) in which the tissue was fixed in 4% PFA/PBS. Generally, the background, nonspecific staining is negligible in tissues processed in this fixative and a wider range of antibodies produce a satisfactory immunohistochemical response. Panels A, B and D represent images in an overlapping field with increasing magnification. Panel C represents staining developed with the anti-human CLR antibody AB9414, and is an equivalent field and magnification to panel B, but developed from an adjacent section of tissue. While staining with the anti-CTR antibody was prominent and confined to putative malignant glioma cells, there was little staining evident that would indicate expression of CLR. The insert (panel C) shows that this antibody does detect CLR-ir in vessel walls.

In panels E and F are shown staining with the anti-GFAP monoclonal antibody MAB3402. Panel E (GFAP-ir) represents a similar field as shown in panel B (CTR-ir) but developed from an adjacent section of tissue. As demonstrated in panel D, GFAP-ir cells have a morphology that is characteristic of malignant glioma cells.

In panels G and H are shown images with staining developed with the anti-CTR antibody MCA2191. In panel H this antibody was pre-incubated with 100-fold molar excess of the antigenic peptide used to raise MCA2191 (see Methods section). CTR-ir cells were stained more intensely in the control (panel G) compared to excess peptide (panel H).

In FIG. 4 are shown images from patients (#1, #3 and #6) at low magnification (A, C [Obj.×20] and E [Obj.×10]) and higher power (B, D and F [Obj.×100]), respectively. All images are taken from tissue stained using MCA2191. Panel G is normal brain tissue adjacent to tumour (not shown) from patient #7 [Obj.×40] and panel H represents staining in tissue from patient #8 [Obj.×100].

In each case CTR-ir was associated with cells that have a morphology, and where evident, a nucleus, characteristic of glioma cells. In most examples (panels B, D and F) the tumour tissue is irregularly organised compared to normal tissue shown in panel G. In contrast CTR-ir cells in panel H appear more regularly organised, which could represent cells in a section further away from the sites of proliferation.

For each patient (n=12) the analysis of adjacent sections stained either with MCA 2191 (CTR-ir) or counter-stained with haematoxylin/eosin, determined the proportion (%) of CTR-ir cells with a morphology characteristic of malignant cells. In ten out of twelve patient samples tested, a majority of cells with a malignant morphology were CTR-ir (n=9, >90%; n=1, 60-70%; n=2, ˜0%). Statistical analysis of these data using the Chi-square analysis on a one way classification (Chi-square=4.0, degree of freedom=1) resulted in p<0.05.

In FIG. 5 the images of multi-labelled immuno-fluorescence generated using confocal microscopy are shown of GBM tissues from patient #13 (panels A-D) and patient #14 (panels E-L). In panel A is shown composite staining in which arrows indicate examples of CTR+/GFAP+/nestin+ cells and arrowheads CTR+/nestin+/GFAP— cells. In panels E and I are shown the composite staining (for panels F, G & H and panels J, K & L, respectively) for representative cells that are CTR+/GFAP+/CD133+.

FIG. 6 illustrates the concentration response curves and immunoblot for the GBM cell line A172. In (A) phosphorylation of ERK1/2 was inhibited with increasing concentrations of hCT (log EC₅₀=−10.5) compared to the level of phosphorylation measured in the presence of 3% serum (100%) The inhibition was inhibited with the inclusion of the antagonist of CTR, namely 10⁻⁶M sCT (8-32). In (B) adenylyl cyclase activity was stimulated with increasing concentrations of hCT (log EC₅₀ was −9.9). The stimulation was inhibited by the antagonist 10⁻⁶M sCT (8-32). In immunoblots (C) of whole cell lysates of A172 probed with the anti-CTR antibody MCA2191, there is a minor band that runs with a mobility of ˜70 kD and a major band at 52 kD.

### Example 3

### Discussion

The present inventors describe the validation of CTR as the major target for anti-CTR antibodies and demonstrate it further in experiments with immunoblots and confocal microscopy of cell lines (COS-7/CTR-positive and COS-7/CTR-negative controls) as illustrated in FIG. 1. These observations were based on the development of highly specific anti-human CTR antibodies by the inventors. In FIG. 1, the immunoblots demonstrated the specific interaction of both antibodies with the major band at approximately 67 kD and minor band equivalent to 52 kD, and are likely to correspond to glycosylated hCTR and an unglycosylated form, respectively. In contrast the cell line A172 expressed predominantly the smaller, unglycosylated form of CTR (52 kD) as well as lesser relative amounts of the glycosylated form (67 kD, FIG. 6).

In the confocal immuno-fluorescence studies (FIG. 1), COS-7/CTR+ control cells displayed binding sites for 9B4 and MCA2191 that appeared concentrated on or near the cell surface, but were absent in the COS-7/CTR− control cell lines. Both cell lines were negative with the IgG2A antibody isotype control. Neutralisation of antibody with immunising peptide prior to IHC markedly reduced staining in glioma cells (FIG. 3, panels G and H). Together these results demonstrate that these antibodies are specific for CTR and CTR is expressed by malignant cells in GBM tissues.

Similar regions in serial sections are shown in FIG. 2, panels A, C and E. In FIG. 2B the rounded morphology of malignant cells is shown at higher magnification. Also characteristic of these cells is a large open nucleus with dense condensations of chromatin and an intense nucleolus. Both anti-CTR antibodies identified similar cells in serial sections (FIG. 2, D and F).

In view of the homology (overall 47% identity in the primary structure although no significant identity in the epitope used to raise MCA2191) that exists between CTR and CLR, the CTR-ir cells were tested but did not stain positively when staining using the anti-CLR antibody (FIG. 3C). On the other hand, the anti-hCLR antibody used here did detect CLR-immuno-reactivity within the walls of some vessels associated with the tumour (insert, FIG. 3C) as well as cells in the periphery of necrotic regions (not shown). Taken together the CTR-ir reflects specifically the expression of human CTR protein rather than CLR.

In several examples the gross microscopic features of these tumours include vascular spaces packed with blood cells, some complex glomeruloid structures (CGS, FIG. 4A) and spaces that appear acellular. Around the perimeter of many of CGS are densely packed elongated malignant cells. Within the zones of proliferation there is considerable regional heterogeneity in terms of subpopulations of cells each with a characteristic nuclear morphology. In some tissue sections there was considerable regional heterogeneity of CTR-ir, which suggests differential expression of CTR.

In GBM, malignant cells express glial fibrilliary associated protein (GFAP) (Colin et al., 2006) as shown in FIG. 3 (E and F) and FIG. 5 and displayed morphologies characteristic of glioma cells, as demonstrated in the sections counter-stained with haematoxylin and eosin (FIG. 2). Furthermore, CTR+/GFAP+ cells also expressed nestin and in some subsets of smaller cells CD133 (FIG. 5), a marker of cancer stem cells or BTICs (brain tumor initiating cells). Many of these CTR-ir cells appeared to have a granular cytoplasm (FIGS. 2D, 3D, 3F, 4D) when viewed under high magnification which would be consistent with the presence of secretory mechanisms. Such secretory products might include growth factors that are considered instrumental in the expansion of tumours (Louis, 2006; Adams and Strasser, 2008; D'Abaco and Kaye, 2008).

CTR-ir was evident in malignant cells in a majority of cases of GBM tumours (FIGS. 2-4, 10 out of 12 positive and in FIG. 5, 2 out of 2 positive) examined in this study. A Chi-square statistical analysis was performed on these data and the significance was p<0.05. In the negative samples the likely reasons for lack of staining may range from inadequate preparation of the tissues or little or no detectable expression of CTR, perhaps the result of a negative region (from heterogeneity) in the sample.

Inflammatory processes play an important role in tumour expansion. In prostate cancer stem cells (CD133+) up-regulation of genes associated with a number of functions including inflammation and metastasis have been described (Birnie et al., 2008). Furthermore, in cell lines derived from prostate cancer the CT/CTR axis has been associated with up-regulation of genes involved in inflammation, cell adhesion and metastasis (Shah et al., 2008). The expression of CTR mRNA is stimulated by pro-inflammatory cytokines TNFα and IL1β in cultures of primary astrocytes (Meeuwsen et al., 2003). It is likely that CTR expression by malignant cells forms a component of the inflammatory processes in GBM. For this reason CTR will be a useful target for anti-tumour expansion as demonstrated in mouse xenograft models of breast cancer (Nakamura et al., 2007) in which administration of calcitonin significantly reduced the tumor volume. In this context, the expression of CTR and activation of intracellular signals will also alter the cell cycle of malignant cells as discussed below.

In normal muscle satellite cells, the expression of CTR is associated with quiescence and in other primary cell types, the migration of cells. In some cancer cell lines, such as T47D, CT inhibits cell proliferation (Iwasaki et al., 1983; Lacroix et al., 1998). In transfected cell lines activation of CTR also slows the cell cycle with arrest at the G2/M transition (Evodokiu et al., 1999; Evodokiu et al., 2000) via a pathway involving p21 and internalization of the receptor. Phosphorylation of CTR provides part of the mechanism for internalization, CTR is a substrate of multiple kinases including ERK and phoshorylation sites have been defined in the intracellular C-terminal domain of CTR (Nygaard et al., 1997; Seck et al., 2005). In FIG. 6 the inhibition of phosphorylation of ERK by increasing concentrations of hCT is demonstrated in the GBM cell line A172. The EC₅₀ is characteristic of an effect mediated by CTR and inhibition was antagonised by the CTR antagonist sCT (8-32).

The finding that in 12/14 cases of GBM studied here many of the glioma cells were CTR-ir while adjoining tissues and structures such as blood vessels were negative and the identification of CTR+/CD133+ cells demonstrates utility for anti-CTR antibodies in diagnostic imaging, to assess minimal residual disease following conventional therapies, in the determination of prognosis and predictive outcomes, and as a therapeutic target.

It will be appreciated by persons skilled in the art that numerous variations and/or modifications may be made to the invention as shown in the specific embodiments without departing from the scope of the invention as broadly described. The present embodiments are, therefore, to be considered in all respects as illustrative and not restrictive.

All publications discussed and/or referenced herein are incorporated herein in their entirety.

The present application claims priority from AU 2010902958, the entire contents of which are incorporated herein by reference.

Any discussion of documents, acts, materials, devices, articles or the like which has been included in the present specification is solely for the purpose of providing a context for the present invention. It is not to be taken as an admission that any or all of these matters form part of the prior art base or were common general knowledge in the field relevant to the present invention as it existed before the priority date of each claim of this application.

### REFERENCES

Adams and Strasser (2008) Cancer Res, 68:4018-4021.
Almeida and Allshire (2005) Trends Cell Biol, 15:251-258.
Bird et al. (1988) Science, 242:423-426.
Birnie et al. (2008) Genome Biol, 9:R83.
Boros et al. (2005) J Org Chem, 24:5331-5334.
Colin et al. (2006) Neuropathology and Applied Neurobiology, 32:189-202.
Copp et al. (1962) Endocrinology, 70:638-649.
D'Abaco and Kaye (2008) J Clin Neurosci, 15:1079-1084.
Dong et al. (2009) J Biol Chem, 284:21839-21847.
Evdokiou et al. (1999) Mol Endocrinol, 13:1738-1750.
Evdokiou et al. (2000) J Mol Endocrinol, 25:195-206.
Goldring et al. (1993) Horm Metab Res, 25:477-480.
Gorn et al. (1995) J Clin Invest, 95:2680-2691.
Greenwood et al. (1993) Eur J Immunol, 23: 1098-1104.
Harmsen and De Haard (2007) Appl Microbiol Biotechnol, 77:13-22.
Hirsch and Baruch (2003) Endocrine, 21:201-208.
Hirsch (1964) Science, 146:412-413.
Hoessli et al. (1983) Proc Natl Acad Sci USA, 80:439-443.
Huston et al. (1988) Proc Natl Acad Sci USA, 85:5879-5883.
Iwasaki et al. (1983) Biochem Biophys Res Commun, 110:235-242.
Jones et al. (1986) Nature, 321:522-525.
Lacroix et al. (1998) Eur J Pharmacol, 344:279-286.
Lin et al. (1991) Science, 254:1022-1024.
Louis (2006) Ann Rev Pathol, 1:97-117.
Masi et al. (1998) Biochem Biophys Res Commun, 245:622-626.
Meeuwsen et al. (2003) Glia, 43:243-253.
Millar and Waterhouse (2005) Funct Integr Genomics, 5:129-135.
Morrison et al. (1984) Proc Natl Acad Sci USA, 81:6851-6855.
Mould and Pondel (2003) Cancer Cell Int, 3:6-12.
Muyldermans (2001) J Biotechnol, 74:277-302.
Nakamura et al. (1997) Hum Genet, 99:38-41.
Nakamura et al (2007) J Mol Endocrinol, 39:375-384.
Nygaard et al. (1997) J Bone Miner Research, 12:1681-1690.
Pasquinelli et al. (2005) Curr Opin Genet Develop, 15:200-205.
Pastan et al. (1986) Cell, 47:641.
Perriman et al. (1992) Gene, 113:157-163.
Saha et al. (1994) Semin Nucl Med, 24:324-349.
Seck et al. (2005) Mol Endicronol, 19:2132-2144.
Schmidt et al. (1991) J Biol Chem, 266:18025-33.
Sha et al. (2008) Endocr Relat Cancer, 15:953-964.
Shippy et al. (1999) Mol Biotech, 12:117-129.
Silvestris et al. (2008) Leuk Res, 32:611-623.
Singh et al. (2003) Cancer Res, 63:5821-5828.
Singh et al. (2004) Nature, 432:396-401.
Sun et al. (1986) Hybridoma 5 Suppl, 1:517-20.
Thorpe et al. (1987) Cancer Res, 47:5924.
Tibary et al. (2007) Soc Reprod Fertil Suppl, 64:297-313.
Tikellis et al. (2003) Kidney International, 63:416-426.
Vitetta et al. (1987) Science, 238:1098.
Waldmann (1991) Science, 252:1657.
Wittig et al. (2006) Nat Protoc, 1:418-428.
Wookey et al. (2008) Histopathol, 52:605-612.
Wookey et al. (2009) Histochem Cell Biol, 132:181-189.

1. A method for the localisation, diagnosis, prognosis, and/or prediction of therapeutic outcome of a brain tumor in a subject, the method comprising detecting calcitonin receptor in brain cells of the subject, wherein the presence of calcitonin receptor localises, is diagnostic, prognostic and/or predictive for, the brain tumor.

2. The method of claim 1, wherein the method comprises administering to the subject a compound that binds calcitonin receptor, allowing the compound to bind to cells within the subject, and determining the location of the compound within the brain of the subject.

3. The method of claim 1, wherein the method comprises detecting calcitonin receptor in a sample obtained from the subject.

4. (canceled)

5. The method of claim 2, wherein the compound is detectably labelled.

6.-7. (canceled)

8. The method of claim 1, wherein the method comprises determining the level of calcitonin receptor in the brain cells of the subject and comparing the level of calcitonin receptor in the brain cells of the subject with a control, wherein a higher level of calcitonin receptor compared to the control localises, is diagnostic, prognostic and/or predictive for, the brain tumor.

9. The method of claim 1 further comprising
administering or recommending a therapeutic for the treatment of the brain tumor.

10. A method for treating or preventing a brain tumor in a subject, the method comprising administering to the subject an effective amount of a compound that binds calcitonin receptor to inhibit the growth of, or kill, brain tumor cells in the subject.

11. The method of claim 10, wherein the compound is conjugated to a cytotoxic agent or biological response modifier.

12.-13. (canceled)

14. The method of claim 2, wherein the compound binds an epitope of calcitonin receptor and the epitope comprises an amino acid sequence selected from SEQ ID NOs: 3, 4 and 5.

15. The method of claim 14, wherein the compound comprises an antibody.

16.-23. (canceled)

24. The method of claim 10, wherein the method is performed in combination with, prior to and/or after treatment with a chemotherapeutic or radiotherapeutic.

25. A method for treating or preventing a brain tumor in a subject, the method comprising administering to the subject an effective amount of a compound that reduces the production and/or activity of calcitonin receptor in brain cells of the subject.

26. The method of claim 25, wherein the compound reduces the level of calcitonin receptor mRNA in the brain cells.

27. The method of claim 25, wherein the method is performed in combination with, prior to and/or after treatment with a chemotherapeutic or radiotherapeutic.

28. The method of claim 26, wherein the compound is selected from an antisense polynucleotide, a catalytic polynucleotide, a microRNA and a dsRNA.

29.-32. (canceled)

33. The method of claim 1, wherein the brain cells are glial cells.

34.-49. (canceled)

50. The method of claim 10, wherein the compound binds an epitope of calcitonin receptor and the epitope comprises an amino acid sequence selected from SEQ ID NOs: 3, 4 and 5.

51. The method of claim 10, wherein the brain cells are glial cells.

52. The method of claim 25, wherein the brain cells are glial cells.


SEQUENCE LISTING (5 sequences)

SEQ ID NO: 1 - DNA, 3470 bp, Homo sapiens
gtgcgcacgtccgcacctcaccctgcggctgacatctcctgcccaggagatgggcgctgaagcttgagcgcctgagtccctggagccacacctgcgaacaccctttgcttctattgagctgtgcccagccgcccagtgacagaattccaggacaaagagatcttcaaaaatcaaaaatgaggttcacatttacaagccggtgcttggcactgtttcttcttctaaatcacccaaccccaattcttcctgccttttcaaatcaaacctatccaacaatagagcccaagccatttctttacgtcgtaggacgaaagaagatgatggatgcacagtacaaatgctatgaccgaatgcagcagttacccgcataccaaggagaaggtccatattgcaatcgcacctgggatggatggctgtgctgggatgacacaccggctggagtattgtcctatcagttctgcccagattattttccggattttgatccatcagaaaaggttacaaaatactgtgatgaaaaaggtgtttggtttaaacatcctgaaaacaatcgaacctggtccaactatactatgtgcaatgctttcactcctgagaaactgaagaatgcatatgttctgtactatttggctattgtgggtcattctttgtcaattttcaccctagtgatttccctggggattttcgtgtttttcaggagccttggctgccaaagggtaaccctgcacaagaacatgtttcttacttacattctgaattctatgattatcatcatccacctggttgaagtagtacccaatggagagctcgtgcgaagggacccggtgagctgcaagattttgcattttttccaccagtacatgatggcctgcaactatttctggatgctctgtgaagggatctatcttcatacactcattgtcgtggctgtgtttactgagaagcaacgcttgcggtggtattatctcttgggctgggggttcccgctggtgccaaccactatccatgctattaccagggccgtgtacttcaatgacaactgctggctgagtgtggaaacccatttgctttacataatccatggacctgtcatggcggcacttgtggtcaatttcttctttttgctcaacattgtccgggtgcttgtgaccaaaatgagggaaacccatgaggcggaatcccacatgtacctgaaggctgtgaaggccaccatgatccttgtgcccctgctgggaatccagtttgtcgtctttccctggagaccttccaacaagatgcttgggaagatatatgattacgtgatgcactctctgattcatttccagggcttctttgttgcgaccatctactgcttctgcaacaatgaggtccaaaccaccgtgaagcgccaatgggcccaattcaaaattcagtggaaccagcgttgggggaggcgcccctccaaccgctctgctcgcgctgcagccgctgctgcggaggctggcgacatcccaatttacatctgccatcaggagctgaggaatgaaccagccaacaaccaaggcgaggagagtgctgagatcatccctttgaatatcatagagcaagagtcatctgcttgaatgtgaagcaaacacagcatcgtgatcactgagccatcatttcctgggagaaagaccatgcatttaaagtattctccatcctcccaggaaccgaacatatcatttgtgaagaattattcagtgaatttgtccattgtaaatctgaagaaagttattcttggtactgttgctttgggagacagtctaggaatggagtctcccactgcaacttgtgaactccatcattcatccaggactgagatgcaaatgtcacagtaatgcaagcaaagtatcaaagaaaaacaatgaaattgacctagttcagatacagggtgctccttgtcaatactgagccatttatacctttgaaatattaaaatcactgtcaatatttttatttttaactctggattttgaattagattatttctgtatttggctatggatctgatttttaatttttttaaatttcagtcaattctgatgttactgagatgttttaccatccttacaatgtaaaccacatgaactacgtgacctctgcaagacaaagcggctttctaatagagagattagtaaatatgtgaagaaaaagacctgcatttggcaggaagatgtatgctttgaatgcaaaagaaatttagagtcaatttgctgaaaacattacatgctcagcttggttttggacaagcctgtccattgggcaggacctagctgttgtaaagaattggtcttaatgttgaatgtattttggttgctgatgtttataaactgagaggtcacaaagaatctatcactaaaaatttttacaaaactgccaaaaatataattcttagtggaagacaatactccctttaaagagagtttgccactcccctaaactccaggatttataaagcaaattactccaaggtttataaagcagattacctcttgcccttgggtgctatctagcagtaaaagataaatttgttgaatattggtaattaaaagactccacataagtccattaactgctttccacccagcttcaaagcttaaaaagagctcaggcttttccaggaagatccaggagggctaattagaaatcaacttgtggttgaccgcttgtttcttgttattaccaaaacaggaggggaaaaaattaactgctccaaatttaaccataaatcaattcatgtttaacgtttctcattaaaatccagtattatattatcatatctctctttacttcccagtataagatttttgaaaatcctgaataaaccagtatcgttactggcacctgaaattaatttgtgaatttgcaacagtaatcagagttaccattatttaatttgtatgctaaatgaggaggtacattgaaaccctccaaatctccagtctcatctatgtcatattttgccactgcctttcagaagtgatttagttgtggaaagataataaattgatttgttatggttacatatttagcgcacccagagaaaattaattatatttctacagagaaaatgaatttgggatactaaagtagtttaagtctcctttactgaatgtaagggggggatcgaaaagaaggtatttttccaatcacagtgttatgtagtattgttctatttttgtttacaaacatggaaaacagagtatttctggcagctgtggtacaaatgtgataatatattgctaaaatattttagatgttattatgctaatatagtaggggttgaagaaaacaaaatagcttattatagaattgcacatagttctgcccaaattatgtgaaatgcttatgcttgtgtatatgtataaattaatacagagtacgttaaaagcaaaaagatgtatatttgcatatttttctaaagaaatatattattcatcttttcattc

SEQ ID NO: 2 - protein, 474 aa, Homo sapiens
MRFTFTSRCLALFLLLNHPTPILPAFSNQTYPTIEPKPFLYVVGRKKMMDAQYKCYDRMQQLPAYQGEGPYCNRTWDGWLCWDDTPAGVLSYQFCPDYFPDFDPSEKVTKYCDEKGVWFKHPENNRTWSNYTMCNAFTPEKLKNAYVLYYLAIVGHSLSIFTLVISLGIFVFFRSLGCQRVTLHKNMFLTYILNSMIIIIHLVEVVPNGELVRRDPVSCKILHFFHQYMMACNYFWMLCEGIYLHTLIVVAVFTEKQRLRWYYLLGWGFPLVPTTIHAITRAVYFNDNCWLSVETHLLYIIHGPVMAALVVNFFFLLNIVRVLVTKMRETHEAESHMYLKAVKATMILVPLLGIQFVVFPWRPSNKMLGKIYDYVMHSLIHFQGFFVATIYCFCNNEVQTTVKRQWAQFKIQWNQRWGRRPSNRSARAAAAAAEAGDIPIYICHQELRNEPANNQGEESAEIIPLNIIEQESSA

SEQ ID NO: 3 - protein, 18 aa, Artificial Sequence
  Epitope 1 of human CTR
DIPIYICHQELRNEPANN

SEQ ID NO: 4 - protein, 17 aa, Artificial Sequence
  Epitope 2 of human CTR
PSEKVTKYCDEKGVWFK

SEQ ID NO: 5 - protein, 12 aa, Artificial Sequence
  Epitope 3 of human CTR
FSNQTYPTIEPK